(19) United States
(12) Patent Application Publication
(10) Pub. No.: US 2009/0087484 A1
(43) Pub. Date: Apr. 2, 2009

(54) Formulation and dosage form for increasing oral bioavailability of hydrophilic macromolecules

(75) Inventors: Liang C. DONG, Sunnyvale, CA (US); Patrick S. L. WONG, Burlingame, CA (US); Vu A. NGUYEN, San Jose, CA (US); Si-Hong Alicia Yum, Belmont, CA (US); Anthony C. CHAO, Cupertino, CA (US); Peter E. Daddona, Menlo Park, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: ALZA CORPORATION, Mountain View, CA (US)

(21) Appl. No.: 11/864,017

(22) Filed: Sep. 28, 2007

(51) Int. Cl. A61K 31/20; A61K 31/70; A61K 38/00; A61K 38/21; A61K 38/43; A61K 47/00; A61K 9/48; A61K 9/52; A61P 1/00

(52) U.S. Cl. 424/452; 424/457; 424/85.7; 424/94.1; 514/12; 514/2; 514/23; 514/558; 514/56; 514/772.4; 514/773; 514/777; 514/784

(57) ABSTRACT

A formulation and dosage form for enhancing the bioavailability of orally administered hydrophilic macromolecules includes a permeation enhancer, a hydrophilic macromolecule, and a carrier such as a nonionic surfactant that exhibits in-situ gelling properties. The formulation is delivered within the GI tract as a liquid having at least some affinity for the surface of the GI mucosal membrane. Once released, it is believed that the liquid formulation spreads across one or more areas of the surface of the GI mucosal membrane, where the carrier of the formulation then transitions into a bioadhesive gel in-situ. As a bioadhesive gel, the formulation presents the hydrophilic macromolecule and the permeation enhancer at the surface of the GI mucosal membrane at concentrations sufficient to increase absorption of the hydrophilic macromolecule through the GI mucosal membrane over a period of time. A dosage form incorporates the formulation and may be designed to provide the controlled release of the formulation within the GI tract over a desired period of time.

### CROSS-REFERENCE TO RELATED APPLICATIONS

This application is a divisional of U.S. application Ser. No. 10/324,154, filed October Dec. 18, 2002, which claims the benefit of U.S. Provisional Application No. 60/343,005 filed Dec. 19, 2001, pursuant to 35 U.S.C. §119(e), the contents of which are incorporated herein by reference.

### BACKGROUND OF THE INVENTION

1. Field of the Invention

The present invention relates to formulations and dosage forms for increasing the oral bioavailability of hydrophilic macromolecules. In particular, the present invention relates to in-situ gelling formulations that increase the oral bioavailability of hydrophilic macromolecules and to dosage forms that facilitate oral administration of such formulations.

2. State of the Art

In terms of patient compliance, oral administration of a therapeutic agent is generally considered far superior to parental administration. This is particularly true where either the nature of the therapeutic agent or the nature of the condition being treated requires multiple daily dosing of the therapeutic agent. Unfortunately, despite their varied and expanding therapeutic applications, hydrophilic macromolecules, such as polypeptides and polysaccharides, have proven exceedingly difficult to successfully administer orally. One challenge faced when attempting the oral administration of hydrophilic macromolecules is the relatively harsh environment of the upper gastrointestinal (GI) tract, which, due to its relatively low pH and the presence of lytic enzymes, tends to degrade hydrophilic macromolecules such that their therapeutic value is compromised. However, even when hydrophilic macromolecules can be protected from degradation in the upper GI tract, their absorption across the mucosal membrane of the GI tract tends to be minimal, resulting in low oral bioavailabilities. The low absorption of hydrophilic macromolecules across the mucosal membrane of the GI tract is generally attributed to their hydrophilicity, large size, and dense charge polarities. Because of their low oral bioavailability, hydrophilic macromolecules generally must be administered parentally (e.g., via subcutaneous, intramuscular, or intravenous injections) in order to achieve a therapeutic effect.

It would, therefore, be highly desirable to provide a formulation and dosage form that enhance the oral bioavailability of hydrophilic macromolecules to the extent that oral dosing of such molecules may be possible. More than one effort to enhance the oral bioavailability of hydrophilic macromolecules has focused on the use of permeation enhancers to increase absorption of a target molecule across the mucosal membrane of the GI tract. For instance, U.S. Pat. No. 5,424,289, assigned to ALZA Corporation of Mountain View, Calif., discloses a formulation for enhancing the bioavailability of human growth hormone (HGH) in the GI tract. The formulation disclosed in the '289 patent includes an oil and a permeation enhancer, and the formulation may be tableted in a solid dosage form. When tested using a flushed and ligated rat ileal model, the formulation taught in the '289 patent achieved an HGH bioavailability of up to 68%. However, the positive results achieved by the formulation disclosed in the '289 patent have proven difficult to reproduce under conditions which more closely simulate oral administration of the formulation in an animal or human subject. Thus, it would be an improvement in the art to provide a formulation and dosage form that more reliably enhance the oral bioavailability of hydrophilic macromolecules.

### SUMMARY OF THE INVENTION

The present invention includes a formulation that provides increased bioavailability of orally administered hydrophilic macromolecules. In order for a permeation enhancer to successfully increase the bioavailability of a hydrophilic macromolecule within the GI tract, the concentration of the permeation enhancer must be maintained above a certain critical level at the surface of the GI mucosal membrane. However, it has been found that conventional formulations including a permeation enhancer and a hydrophilic macromolecule are diluted relatively rapidly after delivery within the GI tract. Because of the dilution of such formulations, the concentration of permeation enhancer is generally reduced below the critical level for the permeation enhancer such that the permeation enhancer is incapable of significantly increasing absorption of the delivered hydrophilic macromolecule. The present invention, however, provides an in-situ gelling formulation that is capable of adhering to the GI mucosal membrane and presenting effective concentrations of a permeation enhancer and a desired hydrophilic macromolecule at the surface of the GI mucosal membrane such that the oral bioavailability of the hydrophilic macromolecule is enhanced.

The formulation of the present invention includes a permeation enhancer, a hydrophilic macromolecule, and a carrier that exhibits in-situ gelling properties, such as a nonionic surfactant. The formulation of the present invention is delivered within the GI tract as a liquid having at least some affinity for the surface of the GI mucosal membrane. Once released, it is believed that the liquid formulation spreads across one or more areas on the surface of the GI mucosal membrane, where the carrier of the formulation then transitions into a bioadhesive gel in-situ. As a bioadhesive gel, the formulation of the present invention not only adheres to the mucosal membrane of the GI tract, but also reduces or minimizes dilution of both the permeation enhancer and the hydrophilic macromolecule included in the formulation by lumenal fluids and secretions. It is believed, therefore, that the formulation of the present invention increases the bioavailability of a given hydrophilic macromolecule by presenting the hydrophilic macromolecule, together with a suitable permeation enhancer, at the surface of the mucosal membrane of the GI tract at concentrations sufficient to increase absorption of the hydrophilic macromolecule through the GI mucosal membrane over a period of time.

Though the formulation of the present invention may be used to administer any desired hydrophilic macromolecule, the formulation of the present invention is particularly useful for the oral administration of polypeptides and polysaccharides. As used herein the term “polypeptide” encompasses any naturally occurring or synthetic hydrophilic compound including two or more amino acid residues. As used herein the term “polysaccharide” encompasses any naturally occurring or synthetic hydrophilic carbohydrate containing three or more simple sugar molecules.

The present invention further includes a dosage form incorporating the formulation of the present invention. The dosage form may be any pharmaceutically acceptable capsule capable of delivering the formulation of the present invention. For example, the dosage form may include a hard or soft gelatin capsule. The dosage form of the present invention is preferably designed to delay release of the formulation until the dosage form has passed through the stomach and at least entered the small intestine. Therefore, the dosage form of the present invention may include an enteric coating designed to target release of the formulation at a desired point within the GI tract. Alternatively, the dosage form of the present invention may include a controlled release delivery device, which offers the flexibility of delivering the formulation of the present invention according to any desired release pattern. For instance, a controlled release dosage form may be designed to deliver the formulation of the present invention at a zero order, ascending, or descending rate within a targeted area of the GI tract.

### BRIEF DESCRIPTION OF THE DRAWINGS

FIG. 1 through FIG. 5 illustrate various views of controlled release dosage forms of the present invention fabricated using hard gelatin capsules.

FIG. 6 and FIG. 7 provide exterior and cross-sectional views of a controlled release dosage form according to the present invention fabricated using a soft gelatin capsule.

FIG. 8 and FIG. 9 provide exterior and cross-sectional views of the controlled release dosage form illustrated in FIG. 6 and FIG. 7 during operation.

FIG. 10 and FIG. 11 illustrate a second controlled release dosage form according to the present invention fabricated using a soft gelatin capsule.

FIG. 12 and FIG. 13 illustrate a third controlled release dosage form according to the present invention fabricated using a soft gelatin capsule.

FIG. 14A through FIG. 14D illustrate a method for forming a sealed exit orifice for a controlled release dosage form of the present invention fabricated using a soft gelatin capsule.

FIG. 15 and FIG. 16 illustrate a controlled release dosage form according to the present invention having a sealed exit orifice fabricated as shown in FIG. 14A through FIG. 14D.

FIG. 17 through FIG. 19 illustrate a second method for forming a sealed exit orifice for a controlled release dosage form of the present invention fabricated using a soft gelatin capsule.

FIG. 20 provides a graph illustrating the viscosity of CREMOPHOR® EL (ethoxylated castor oil), an exemplary carrier, as a function of water content measured by a Haake Rheometer at 158 rad/s and 37° C.

FIG. 21 provides a graph showing the G′(storage modulus), G″ (loss modulus), and 6 (G″/G′) of various CREMOPHOR® EL/water blends measured by a Haake Rheometer at 158 rad/s and 37° C.

FIG. 22 provides a graph illustrating the dynamic viscosity of various CREMOPHOR® EL/water blends measured by a Haake Rheometer at 37° C.

FIG. 23 provides a graph illustrating the adhesion of various CREMOPHOR® EL/water blends.

FIG. 24 provides a graph illustrating the plasma concentration profile of pentosan polysulfate sodium (PPS) achieved in a flush/ligated (F/L) rat ileal model using various formulations according to the present invention. The error bars on the graph represent the standard deviation of four runs.

FIG. 25 provides a graph illustrating the percent bioavailability of PPS achieved in a F/L rat ileal model using various formulations according to the present invention. The error bars on the graph represent the standard deviation of four runs.

FIG. 26 provides a graph illustrating the plasma concentration profile of PPS achieved in a non-flushed/non-ligated (NF/NL) rat ileal model using various formulations according to the present invention. The error bars on the graph represent the standard deviation of at least three runs.

FIG. 27 provides a graph illustrating the percent bioavailability of PPS achieved in a NF/NL rat ileal model using various formulations according to the present invention, with the error bars representing the standard deviation of at least three runs.

FIG. 28 provides a graph illustrating the effects of permeation enhancer dose on plasma concentration of PPS using various formulations according to the present invention delivered using an NF/NL rat ileal model. The error bars on the graph represent the standard deviation of at least three runs.

FIG. 29 provides a graph illustrating the effects that formulation dose has on the percent bioavailability achieved by various PPS formulations according to the present invention, which were administered using both F/L and NF/NL rat ileal models. The error bars on the graph represent the standard deviation of at least three runs.

FIG. 30 provides a graph describing the plasma concentration profile and percent bioavailability of PPS achieved by various formulations according to the present invention including sodium caprate as a permeation enhancer, each of the formulations being administered using an NF/NL rat ileal model. The error bars on the graph represent the standard deviation of at least three runs.

FIG. 31 provides a graph describing the plasma concentration profile and percent bioavailability of PPS achieved by various formulations of according to the present invention including propylene glycol laurate (PGL) as a viscosity reducing agent, each of the formulations being administered using an NF/NL rat ileal model. The error bars on the graph represent the standard deviation of at least three runs.

FIG. 32 provides a graph describing the plasma concentration profile and percent bioavailability of PPS achieved in dogs as a result of oral administration of a PPS formulation according to the present invention. The error bars on the graph represent the standard deviation of at least three runs.

FIG. 33 provides a graph illustrating the in-vitro release pattern of a formulation according to the present invention as delivered by an enteric coated dosage form according to the present invention.

FIG. 34 provides a graph illustrating the percent bioavailability of unfractionated heparin achieved using a formulation according to the present invention administered using a F/L rat ileal model. The error bars on the graph represent the standard deviation of three runs.

FIG. 35 and FIG. 36 provide graphs illustrating the percent bioavailability of unfractionated heparin achieved using different formulations according to the present invention administered using an NF/NL rat ileal model. The error bars on the graphs represent the standard deviation of three runs.

FIG. 37 provides a graph describing the plasma concentration profile and percent bioavailability of low molecular weight heparin (LMWH) achieved using a formulation according to the present invention administered using a NF/NL rat ileal model. The error bars on the graph that correspond to the saline solution and the i.v. dose represent the standard deviation of three runs, while the error bars on the graph for the gelling formulation represent the standard deviation of five runs.

FIG. 38 provides a graph describing the plasma concentration profile and percent bioavailability of Desmopressin (dDAVP) achieved using various formulations according to the present invention, each of the formulations being administered using a NF/NL rat ileal model. The error bars on the graph represent the standard deviation of three runs.

FIG. 39 provides two graphs illustrating the stability of dDAVP over time when included in a formulation according to the present invention, with the first graph illustrating the stability of dDAVP in a formulation that does not include an antioxidant and the second graph illustrating the stability of dDAVP in a formulation including butylated hydroxytoluene (BHT) as an antioxidant.

FIG. 40 provides a graph illustrating the release profiles of dDAVP achieved using different dosage forms according to the present invention incorporating dDAVP formulations.

FIG. 41 provides a graph describing the plasma concentration profiles and percent bioavailabilities of dDAVP achieved using different dosage forms according to the present invention. The error bars on the graph represent the standard deviation of three runs

### DETAILED DESCRIPTION OF THE INVENTION

The formulation of the present invention includes a hydrophilic macromolecule, a permeation enhancer, and a carrier that exhibits in-situ gelling properties. The formulation of the present invention may also include a viscosity reducing agent to further facilitate spreading of the formulation across the surface of the mucosal membrane of the GI tract. The precise amounts of each component of the formulation of the present invention will vary according to several factors. Among such factors are the particular hydrophilic macromolecule to be delivered, the condition to be treated, and the nature of the subject. However, in each instance, the amount of each compound of the formulation of the present invention is chosen to facilitate delivery of an amount of the hydrophilic macromolecule sufficient to provide a therapeutic effect to the subject.

The hydrophilic macromolecule included in the formulation of the present invention generally comprises about 0.01 wt % to about 50 wt % of the formulation. Though the formulation of the present invention may incorporate any hydrophilic macromolecules providing a therapeutic benefit, the formulation of the present invention is particularly useful for the oral administration of therapeutic polypeptides and polysaccharides. Specific polypeptides that may be included in the formulation of the present invention include, but are not limited to, insulin, human growth hormone, IFN-α, samon calcitonin, erythropoietin (EPO), TPA (Activase), G-CSF (Neupogen), Factor VIII (Kogenate), growth hormone-releasing peptide, O-casomorphine, renin inhibitor, tetragastrin, pepstatinylglycine, leuprolide, empedopeptin, β-lactoglobulin, TRH analogues, ACE inhibitors, and cyclosporine. Exemplary polysaccharides that may be included in the formulation of the present invention include, but are not limited to, pentosan polysulfate sodium (PPS), unfractionated heparin, and low molecular weight heparin (LMWH). In addition, the formulation of the present invention may include more than one different hydrophilic macromolecule. Where more than one hydrophilic macromolecule is incorporated into the formulation of the present invention, the combined weight percent of the included hydrophilic macromolecules accounts for between about 0.01 wt % and 50 wt % of the formulation.

The specific amount of hydrophilic macromolecule included in the formulation of the present invention will vary according to the nature of the macromolecule, the dose of hydrophilic macromolecule needed, the dose of formulation administered, and the bioavailability of the macromolecule when delivered using the formulation of the present invention. In each instance, however, the formulation of the present invention will include an amount of hydrophilic macromolecule sufficient to create and maintain a concentration gradient across the GI mucosal membrane such that the absorption of the hydrophilic macromolecule is increased.

The permeation enhancer included in the formulation of the present invention may include any entity that is compatible with the formulation of the present invention and enhances absorption of the chosen hydrophilic macromolecule across the mucosal membrane of the GI tract. Permeation enhancers suitable for use in the formulation of the present invention include, but are not limited to, ethylene-diamine tetra-acetic acid (EDTA), bile salt permeation enhancers, such as sodium deoxycholate, sodium taurocholate, sodium deoxycholate, sodium taurodihydrofusidate, sodium dodecylsulfate, sodium glycocholate, taurocholate, glycocholate, taurocheno-deoxycholate, taurodeoxycholate, deoxycholate, glycodeoxycholate, and ursodeoxycholate, fatty acid permeation enhancers, such as sodium caprate, sodium laurate, sodium caprylate, capric acid, lauric acid, and caprylic acid, acyl carnitines, such as palmitoyl carnitine, stearoyl carnitine, myristoyl camitine, and lauroyl carnitine, and salicylates, such as sodium salicylate, 5-methoxy salicylate, and methyl salicylate. Permeation enhancers generally open the tight junctions formed between epithelial cells of the GI mucosal membrane, and thereby allow diffusion of hydrophilic macromolecules into the intestinal mucosa (i.e., pericellular absorption. Though the amount of permeation enhancer included in the formulation of the present invention will generally range between about 11 wt % and about 30 wt %, the nature and precise amount of permeation enhancer included in the formulation of the present invention will vary depending on, for example, the anticipated subject, the hydrophilic macromolecule to be delivered, the nature of the permeation enhancer itself, and the dose of formulation to be administered.

It has been generally found that the performance of the permeation enhancer is critically dependent upon the concentration of permeation enhancer present at or near the surface of the GI mucosal membrane. Therefore, the amount of permeation enhancer included in the formulation should be sufficient to maintain an effective concentration of permeation enhancer (i.e., a concentration above the critical concentration for the permeation enhancer used) at or near the surface of the GI mucosal membrane over a period of time sufficient to increase the bioavailability of the hydrophilic macromolecule. Where possible, the permeation enhancer can be chosen such that the permeation enhancer not only facilitates absorption of the chosen hydrophilic macromolecule, but also resists dilution by lumenal fluids or secretions.

The carrier of the formulation of the present invention allows the formulation to transition from a relatively non-adhesive, low viscosity liquid to a relatively viscous, bioadhesive gel after the formulation has been delivered within the GI tract of a subject. The carrier of the formulation of the present invention is chosen such that the transition from a relatively non-adhesive, low viscosity liquid to a relatively viscous, bioadhesive gel occurs after the formulation has been released within the GI tract and had some opportunity to arrive at the surface of the GI mucosal membrane. Hence, the carrier of the formulation of the present invention enables the in-situ transition of the formulation from a liquid to a bioadhesive gel. Due to its high viscosity and bioadhesive properties, the gel formed by the formulation of the present invention holds the permeation enhancer and the hydrophilic macromolecule together at the surface of the GI mucosal membrane and protects both such components from dilution and enzymatic degradation over a period of time.

Suitable carriers that exhibit in-situ gelling properties include non-ionic surfactants that transition from a relatively non-adhesive, low viscosity liquid to a relatively viscous, bioadhesive liquid crystal state as they absorb water. Specific examples of non-ionic surfactants that may be used as the carrier in the formulation of the present invention include, but are not limited to, Cremophor (e.g., CREMOPHOR® EL and CREMOPHOR® RH), Incordas 30, polyoxyethylene 5 castor oil, polyethylene 9 castor oil, polyethylene 15 castor oil, d-α-tocopheryl polyethylene glycol succinate (TPGS), monoglycerides, such as myverol, aliphatic alcohol based nonionic surfactants, such as oleth-3, oleth-5, polyoxyl 10 oleyl ether, oleth-20, steareth-2, stearteth-10, steareth-20, ceteareth-20, polyoxyl 20 cetostearyl ether, PPG-5 ceteth-20, and PEG-6 capryl/capric triglyceride, Pluronic® and tetronic block copolymer non-ionic surfactants, such as Pluronic® L10, L31, L35, L42, L43, L44, L62, L61, L63, L72, L81, L101, L121, and L122, polyoxylene sorbitan fatty acid esters, such as Tween 20, Tween 40, Tween 60, Tween 65, Tween 80, Tween 81, and Tween 85, and ethoxylated glycerides, such as PEG 20 almond glycerides, PEG-60 almond glycerides, PEG-20 corn glycerides, and PEG-60 corn glycerides. Generally, the carrier of the formulation of the present invention will account for about 35 wt % to about 88 wt % of the formulation. Of course, the specific type and amount of carrier included in the formulation of the present invention may vary depending on, among other factors, the anticipated subject, the hydrophilic macromolecule to be delivered, the permeation enhancer chosen, and the amount of hydrophilic macromolecule to be delivered across the mucosal membrane of the GI tract.

Where a non-ionic surfactant is used as the carrier of the formulation of the present invention, the initial viscosity of the formulation (i.e., the viscosity exhibited by the formulation as it is delivered within the GI tract) and the time required for the formulation to transition to a bioadhesive gel can be at least partially controlled through the addition of water. As water is added to a formulation having a non-ionic surfactant as the carrier, the initial viscosity of the formulation will increase. However, as water content increases, the increase in viscosity of nonionic surfactants tends to be non-linear. Often, as the water content of a nonionic surfactant exceeds a certain threshold, the viscosity of the nonionic surfactant increases rapidly as the nonionic surfactant transitions to its gelling state. Thus, control of the initial viscosity of a formulation including a nonionic surfactant carrier may be limited. Nevertheless, because nonionic surfactants tend to exhibit such a threshold behavior, the time required by a nonionic surfactant carrier to transition into a bioadhesive gel can be controlled, at least in part, by including greater or lesser amounts of water in the formulation. If a relatively quick conversion is desired, a formulation including a nonionic surfactant may be provided more water, thereby placing the formulation closer to the water content threshold at which the formulation will rapidly convert to a bioadhesive gel. In contrast, if a relatively slow conversion is desired, the formulation may include less water or no water, thereby placing the formulation farther from the gelling threshold.

The formulation of the present invention may also include a viscosity reducing agent that reduces the initial viscosity of the formulation. Reducing the initial viscosity of the formulation may further facilitate spreading of the formulation of the present invention across one or more areas of the GI mucosal membrane after the formulation is delivered within the GI tract but before the formulation transitions into a bioadhesive gel. Exemplary viscosity reducing agents that may be used in the formulation of the present invention include, but are not limited to, polyoxyethylene 5 castor oil, polyoxyethylene 9 castor oil, labratil, labrasol, capmul GMO (glyceryl mono oleate), capmul MCM (medium chain mono- and diglyceride), capmul MCM C8 (glyceryl mono caprylate), capmul MCM C10 (glyceryl mono caprate), capmul GMS-50 (glyceryl mono stearate), caplex 100 (propylene glycol didecanoate), caplex 200 (propylene glycol dicaprylate/dicaprate), caplex 800 (propylene glycol di 2-ethyl hexanoate), captex 300 (glyceryl tricapryl/caprate), captex 1000 (glyceryl tricaprate), captex 822 (glyceryl triandecanoate), captex 350 (glyceryl tricaprylate/caprate/laurate), caplex 810 (glyceryl tricaprylate/caprate/linoleate), capmul PG8 (propylene mono caprylate), propylene glycol, and propylene glycol laurate (PGL). Where a viscosity reducing agent is included in the formulation of the present invention, the viscosity reducing agent will generally account for up to about 10 wt % of the formulation. As is true of each of the other constituents of the formulation of the present invention, however, the precise amount of viscosity reducing agent included in the formulation of the present invention may be varied, as desired, to achieve a sought after therapeutic benefit.

The capability of the formulation of the present invention to transition from a relatively non-adhesive, low viscosity liquid to a viscous, bioadhesive gel in-situ is believed to impart functional advantages to the formulation of the present invention, relative to simply delivering the formulation as a bioadhesive gel. For example, it is believed that delivering the formulation as a relatively non-adhesive, low viscosity liquid enables the formulation to more easily spread across one or more areas of the GI mucosal membrane before converting to a relatively viscous, bioadhesive gel. This would allow a given volume of the formulation to present the hydrophilic macromolecule and permeation enhancer over a greater area of the GI mucosal membrane, thereby increasing the amount of hydrophilic macromolecule absorbed for a given volume of formulation. Another advantage imparted by delivering the formulation of the present invention as a relatively non-adhesive, low viscosity liquid is that doing so is believed to reduce indiscriminant adhesion of the formulation of the present invention to material contained within the GI lumen. As is easily appreciated, if the formulation was delivered as a bioadhesive substance, the formulation could indiscriminately adhere to the lumenal contents instead of the GI mucosal membrane, limiting the amount of formulation available to adhere to the GI mucosal membrane. In extreme instances, if the formulation was delivered as a bioadhesive substance, the entire volume of the formulation delivered may be encapsulated by or adhere to lumenal contents before the formulation had the opportunity to adhere to the mucosal membrane of the GI tract, and in such instances the intended benefits of the formulation would be entirely negated.

In order to enhance the stability of the formulation of the present invention, the formulation may include an antioxidant or a preservative. For example, an antioxidant may be used to increase the long-term stability of the hydrophilic macromolecule included in the formulation. Specific examples of antioxidants suitable for use in the formulation of the present invention include, for example, butylated hydroxytoluene (BHT), ascorbic acid, fumaric acid, malic acid, ∝-tocopherol, ascorbic acid palmitate, butylated hydroxyanisole, propyl gallate, sodium ascorbate, and sodium metabisulfate. In addition, an antioxidant or preservative included the formulation of the present invention may stabilize more than one constituent of the formulation. Alternatively, the formulation of the present invention may include more than one different preservative or antioxidant, each preservative or antioxidant stabilizing one or more different components of the formulation.

The present invention also includes a dosage form for oral administration of the formulation of the present invention. The dosage form of the present invention contains the formulation of the present invention and must be capable of delivering the formulation of the present invention as desired within the GI tract of the intended subject. In order to preserve the therapeutic efficacy of the hydrophilic macromolecule included in the formulation of the present invention, the dosage form of the present invention is preferably designed to deliver the formulation at a point beyond the upper GI tract. For example, a dosage form according to the present invention may include an enteric-coated gelatin or hydroxypropylmethylcellulose (HPMC) capsule. Enteric coatings will remain intact in the stomach, but will start dissolving once they arrive at the small intestine, thereafter releasing their contents at one or more sites downstream in the intestine (e.g., the ileum and the colon). Enteric coatings are known in the art and are discussed at, for example, Remington's Pharmaceutical Sciences, (1965), 13^{th} ed., pages 604-605, Mack Publishing Co., Easton, Pa.; Polymers for Controlled Drug Delivery, Chapter 3, CRC Press, 1991; Eudragit® Coatings Rohm Pharma, (1985); and U.S. Pat. No. 4,627,851.

If desired, the thickness and chemical constituents of an enteric coating formed on a dosage form of the present invention may be selected to target release of the formulation of the present invention within a specific region of the lower GI tract. Materials suitable for forming enteric coatings for the dosage forms of the present invention include, for example, materials selected from the following groups: (a) phthalate materials, such as cellulose acetyl phthalate, cellulose diacetyl phthalate, cellulose triacetyl phthalate, cellulose acetate phthalate, hydroxypropyl methycellulose phthalate, sodium cellulose either phthalate, celluslose ester phthalate, methycellulose phthalate, cellulose ester-ethry phthalate, alkaline earth salts of cellulose acetate phthalate, calcium salt of cellulose acetate phthalate, ammonium salt of hydroxypropyl methycellulose phthalate, calcium salt of cellulose acetate phthalate, cellulose acetate hexahydrophthalate, hydroxypropyl methylcellulose hexahydrophthalate, or polyvinylacetate phthalate; (b) keartin, deratin, sanaractolu, salol, salol betanapthyl benzoate and acetotannin, salol with balsam of Peru, salol with tolu, salol with gum satic, salol and stearic acid, and salol and shellac; (c) formalized gelatin, and formalized cross-linked gelatin and exchange resins; (d) myristic acid-hydrogenated castor oil-cholesterol, stearic acid-mutton tallow, stearic acid-balsam of tolu, and stearic acid-castor oil; (e) shellac, ammoniated shellac, ammoniated shellac-salol, shellac wool fat, shellac-acetyl alcohol, chellac-stearch acid-balsam of tolu, and shellac n-butyl stearate; (f) abietic acid, methyl abietate, benzoin, balsam of tolu, sandrac, mastic with tolu, and mastic with acetyl alcohol; (g) cellulose acetate phthalate with shellac, start acetate phthalate, polyvinyl acid phthalate, 2-ethocy-5-(2-hydroxyethyxy)-methylcellulose phthlaic acid, acid phthalates of carbohydrates, zein, alkylresin unsaturated fatty acids-shellac, colophony, mixtures of zein and carboxymethylcellulose phthalate; and (h) anionic polymers synthesized from methacrylic acid and methacrylic acid methyl ester, copolymeric acrylic resins of mehacrylic acid and methacrylic acid methyl ester with diallyl phthalates, copolymers of methacrylic acid and methacrylic acid methyl ester with dibutyl phthalate.

Additionally, the dosage form of the present invention may be designed as a controlled release dosage form including an enteric-coated, controlled release delivery device. A controlled release dosage form according to the present invention may provide, for example, a zero order, ascending, descending, or pulsatile rate of formulation release over a period of time ranging from between about 2 hours to about 24 hours. Of course, the delivery period provided by the dosage form of the present invention may be varied as desired and may fall outside the presently preferred range of about 2 hours to about 24 hours.

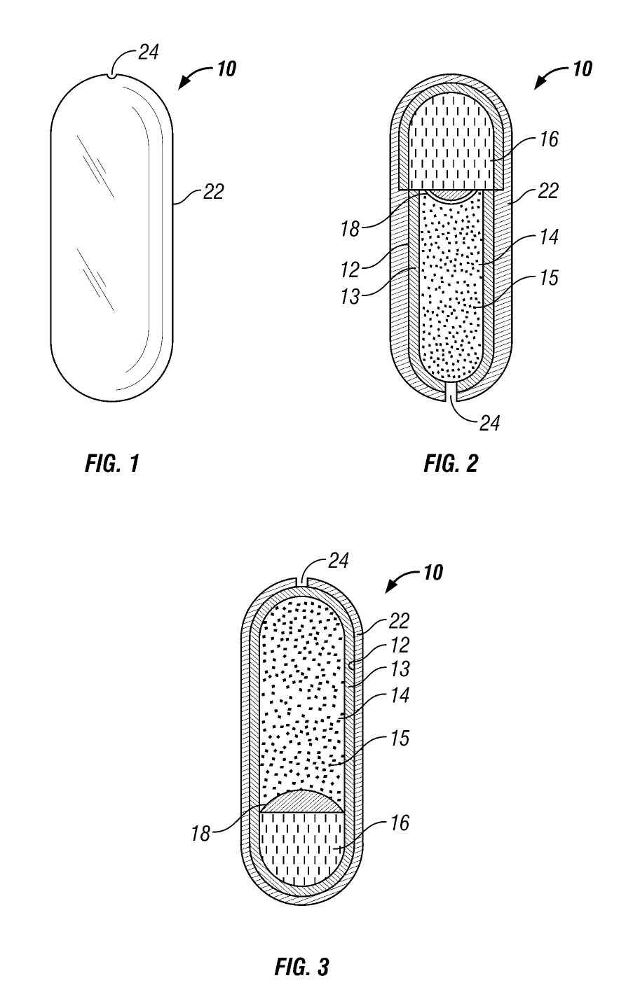

FIG. 1 through FIG. 5 illustrate various controlled release dosage forms 10 according to the present invention that utilize hard pharmaceutical capsules 12 (“hard-caps”). Where a hard-cap 12 is used to create a controlled release dosage form 10 according to the present invention, the hard-cap 12 will include a formulation 14 according to the present invention including a hydrophilic macromolecule 15, and to expel the formulation 14, the hard-cap 12 may also include an osmotic engine 16. Preferably, the osmotic engine 16 and formulation contained in a hard-cap controlled release dosage form 10 of the present invention are separated by a barrier layer 18 that is substantially fluid impermeable. A hard-cap controlled release dosage form 10 of the present invention will generally be coated with a semipermeable membrane 22 and may further include an enteric coating (not illustrated), as already described. In order to facilitate delivery of the formulation 14 from a hard-cap controlled release dosage form 10 of the present invention, the dosage form 10 may include an exit orifice 24, and where provided, the exit orifice 24 may only extend through the semipermeable membrane 22, or, alternatively, the exit orifice 24 may extend down through the wall 13 of the hard-cap 12. If necessary to limit or prevent undesired leakage of the formulation 14, the exit orifice 24 may be sealed using a closure 26.

Any suitable hard-cap may be used to fabricate a controlled release dosage form 10 according to the present invention. For example, U.S. Pat. No. 6,174,547, the contents of which are incorporated herein by this reference, teaches various controlled release hard-cap dosage forms including two-piece or one-piece hard-caps that are suitable for use in the fabrication of a hard-cap controlled release dosage form according to the present invention. Moreover, U.S. Pat. No. 6,174,547 teaches various techniques useful for manufacturing two-piece and one-piece hard-caps. Materials useful for the manufacture of hard-caps useful in a dosage form according to the present invention include, for example, those materials described in U.S. Pat. No. 6,174,547, as well as other commercially available materials including gelatin, a thiolated gelatin, gelatin having a viscosity of about 15 to about 30 millipoise and a bloom strength of up to 150 grams, gelatin having a bloom value of 160 to 250, a composition comprising gelatin, glycerine, water and titanium dioxide, a composition comprising gelatin, erythrosine, iron oxide, and titanium dioxide, a composition comprising gelatin, glycerine, sorbitol, potassium sorbate, and titanium dioxide, a composition comprising gelatin, acacia, glycerin and water, and water soluble polymers that permit the transport of water there through and can be made into capsules.

The osmotic engine 16 of a hard-cap controlled release dosage 10 form of the present invention includes composition that expands as it absorbs water, thereby exerting a push-driving force against the formulation 14 and expelling the formulation 14 from the dosage form 10. The osmotic engine 16 includes a hydrophilic polymer capable of swelling or expanding upon interaction with water or aqueous biological fluids. Hydrorphilic polymers are known also as osmopolymers, osmogels, and hydrogels, and will create a concentration gradient across the semipermeable membrane 22, whereby aqueous is imbibed into the dosage form 10. Hydrophilic polymers that may be used to fabricate an osmotic engine 16 useful in a controlled release dosage form 10 of the present invention include, for example, poly(alkylene oxides), such as poly(ethylene oxide), having weight average molecular weights of about 1,000,000 to about 10,000,000 and alkali carboxymethylcelluloses, such as sodium carboxymethylcellulose, having weight average molecular weights of about 10,000 to about 6,000,000. The hydrophilic polymers used in the osmotic engine 16 may be noncross-linked or cross-linked, with cross-linkages created by covalent or ionic bonds or residue crystalline regions after swelling. The osmotic engine 16 generally includes about 10 mg to about 425 mg of hydrophilic polymer. The osmotic engine 16 may also include about 1 mg to about 50 mg of a poly(cellulose), such as, for example hydroxyethylcellulose, hydroxypropylcellulose, hydroxypropylmethylcellulose, and hydroxypropylbutylcellulose. Further, the osmotic engine 16 may include about 0.5 mg to about 75 mg an osmotically effective solute, such as a salt, acid, amine, ester or carbohydrate selected from magnesium sulfate, magnesium chloride, potassium sulfate, sodium sulfate, lithium sulfate, potassium acid phosphate, mannitol, urea, inositol, magnesium succinate, tartaric acid, sodium chloride, potassium chloride, raffinose, sucrose, glucose, lactose, and sorbitol. Where included, an osmotically effective solute works to imbibe fluid through the semipermeable membrane 22 and into the dosage form 10. Optionally, the osmotic engine 16 may include 0 wt % to 3.5 wt % of a colorant, such as ferric oxide. The total weight of all components in the osmotic engine 16 is equal to 100 wt %. Of course, the osmotic engine 16 included in a controlled release dosage form according to the present invention is not limited to the exact components or the precise component weights described herein. Where included, the osmotic engine 16 is simply formulated to imbibe water into the dosage form 10 and provide a push-driving force sufficient to expel the formulation 14 as water is imbibed and the osmotic engine 16 expands.

Additional hydrophilic polymers that may be used in the osmotic engine 16 of a controlled release dosage form 10 of the present invention include: poly-(hydroxyalkyl methacrylate) having a weight average molecular weight of from 20,000 to 5,000,000; poly(vinylpyrrolidone) having a weight average molecular weight of from 10,000 to 360,000; anionic and cationic hydrogels; polyelectrolyte complexes; poly(vinyl alcohol) having a low acetate residual, cross-linked with glyoxal, formaldehyde, or glutaraldehyde and having a degree of polymerization of from 200 to 300,000; a mixture of methyl cellulose, cross-linked agar and carboxymethyl cellulose; a mixture of hydroxypropyl methycellulose and sodium carboxymethylcellulose; a mixture of hydroxypropyl ethycellulose and sodium carboxymethyl cellulose; sodium carboxymethylcellulose; postassium carboxymethylcellulose; a water insoluble, water swellable copolymer from a dispersion of finely divided copolymer of maleic anhydride with styrene, ethylene, propylene, butylenes, or isobutylene cross-linked with from 0.001 to about 0.5 miles of saturated cross-linking agent per mole of maleic anhydride per copolymer; water swellable polymers of N-vinyl lactams; polyoxyethylene-polyoxypropylene gel; polyoxybutylene-polyethylene block copolymer gel; carbo gum; polyacrylic gel; polyester gel; polyuria gel; polyether gel; polyamide gel; polycellulosic gel; polygum gel; initially dry hydrogels that imbibe and absorb water which penetrates the glass hydrogel and lowers its glass temperature; Carbopol® acidic carboxypolymer, a polymer of acrylic and cross-linked with a polyallyl sucrose, which also known as carboxypolymethylene and carboxyvinyl polymer having a weight average molecular weight of 250,000 to 4,000,000; Cyanamer® polyacrylamides; cross-linked water swellable indene-maleic anhydride polymers; Good-rite® polyacrylic acid having a weight average molecular weight of 100,000; Polyox® polyethylene oxide polymer having a weight average molecular weight of 100,000 to 7,500,000 or higher; starch graft copolymers; and Aqua-Keps® acrylate polymer polysaccharides composed of condensed glucose units such as dieters cross-linked polygluran. Further hydrophilic polymers suitable for use in a controlled release dosage form of the present invention are taught in U.S. Pat. No. 3,865,108, U.S. Pat. No. 4,002,173, U.S. Pat. No. 4,207,893, and Handbook of Common Polymers, Scott and Roff, CRC Press, Cleveland, Ohio, 1971.

Where a barrier layer 18 is provided between the osmotic engine 16 and the formulation 14, the barrier layer 18 works to minimize or prevent mixing of the formulation 14 and the osmotic engine 16 composition before and during operation of the dosage form 10. By minimizing or preventing mixing between the osmotic engine 18 and the formulation 14, the barrier layer 18 serves to reduce the amount of residual formulation 14 that remains within the dosage form 10 once the osmotic engine 18 has ceased expansion or has filled the interior of the dosage form 10. The barrier layer also serves to increase the uniformity with which the driving power of the osmotic engine 18 is transferred to the formulation 14 included in the dosage form 10. The barrier layer is made of a substantially fluid impermeable composition, such as a polymeric composition, a high density polyethylene, a wax, a rubber, a styrene butadiene, a polysilicone, a nylon, Teflon®, a polystyrene, a polytetrafluoroethylene, halogenated polymers, a blend of a microcrystalline, high acetyl cellulose, or a high molecular weight fluid impermeable polymer.

The semipermeable membrane 22 included on a controlled release dosage form 10 of the present invention is permeable to the passage of fluid, such as the aqueous biological fluid present within the GI tract of an animal or human subject, but the semipermeable membrane 22 is substantially impermeable to the passage of the formulation 14 included in the dosage form 10. The semipermeable membrane 22 is non-toxic and maintains its physical and chemical integrity during the drug delivery device of dosage form 10. Further, adjusting the thickness or chemical make-up of the semipermeable membrane 22 can control the release rate or release rate profile provided by a controlled release dosage form 10 according to the present invention. Though the semipermeable membrane 22 may be formed using any suitable material, the semipermeable membrane will generally be formed using materials that include semipermeable polymers, semipermeable homopolymers, semipermeable copolymers, and semipermeable terpolymers. Semipermeable polymers are known in the art, as exemplified by U.S. Pat. No. 4,077,407, and they can be made by procedures described in Encyclopedia of Polymer Science and Technology, Vol. 3, pages 325 to 354, 1964, published by Interscience Publishers, Inc., New York.

Cellulosic polymer materials are well suited for use in forming a semipermeable membrane 22 applied to a controlled release dosage form 10 of the present invention. Where they are used to form a semipermeable membrane 22, cellulosic polymers preferably have a degree of substitution (D.S.) on their anhydroglucose unit ranging from between greater than 0 up to 3 inclusive. As used herein, “degree of substitution” signifies the average number of hydroxyl groups originally present on the anhydroglucose unit that are replaced by a substituting group, or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkylsulfamate, and semipermeable polymer forming groups.

Cellulosic polymers that may be used to form a semipermeable membrane 22 for a controlled release dosage form 10 of the present invention include, for example, cellulose esters, cellulose ethers, and cellulose ester-ethers. Typically, a cellulosic polymer used to create a semipermeable membrane 22 of a controlled release dosage form 10 of the present invention will be selected from the group including cellulose acylate, cellulose diacylate, cellulose triacetate, cellulose acetate, cellulose diacetate, cellulose triacetate, mono-, di-, and tri-cellulose alkanylates, mono-, di-, and tri-alkenylates, mono-, di, and tri-aroylates, and the like. Specific cellulosic polymer materials that may be used to form the semipermeable membrane 22 of a controlled release dosage form 10 of the present invention include, but are not limited to, the following: polymers include cellulose acetate having a D.S. of 1.8 to 2.3 and an acetyl content of 32 to 39.9%; cellulose diacetate having a D.S. of 1 to 2 and an acetyl content of 21 to 35%; and cellulose triacetate having a D.S. of 2 to 3 and an acetyl content of 34 to 44.8%; cellulose propionate having a D.S. of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45% and a hydroxyl content of 2.8 to 5.4%; cellulose acetate butyrate having a D.S. of 1.8, an acetyl content of 13 to 15%, and a butyryl content of 34 to 39%; cellulose acetate butyrate having an acetyl content of 2 to 29.5%, a butyryl content of 17 to 53%, and a hydroxyl content of 0.5 to 4.7%; cellulose triacylates having a D.S. of 2.9 to 3 such as cellulose trivalerate, cellulose trilaurate, cellulose tripalmitate, cellulose trioctanoate, and cellulose tripropionate; cellulose diesters having a D.S. of 2.2 to 2.6 such as cellulose disuccinate, cellulose dipalmitate, cellulose dioctanoate, and cellulose dicarpylate; and mixed cellulose esters such as cellulose acetate valerate, cellulose acetate succinate, cellulose propionate succinate, cellulose acetate octanoate, cellulose valerate palmitate, cellulose acetate heptonate.

Additional semipermeable polymers that may be used to form a semipermeable membrane 22 included on a controlled release dosage form 10 of the present invention include the following: cellulose acetaldehyde dimethyl acetate; cellulose acetate ethylcarbamate; cellulose acetate methylcarbamate; cellulose dimethylaminoacetate; semipermeable polyamides; semipermeable polyurethanes; semipermeable sulfonated polystyrenes; cross-linked, selectively semipermeable polymers formed by the coprecipitation of a polyanion and a polycation as disclosed in U.S. Pat. Nos. 3,173,876, 3,276,586, 3,541,005, 3,541,006, and 3,546,142; semipermeable polymers disclosed by Loeb and Sourirajan in U.S. Pat. No. 3,133,132; semipermeable polystyrene derivatives; semipermeable poly(sodium styrenesulfonate); semipermeable poly(vinylbenzyltrimethyl)ammonium chloride; and semipermeable polymers exhibiting a fluid permeability of 10 to 10 (cc.mil/cm.hr.atm) expressed as per atmosphere of hydrostatic or osmotic pressure difference across a semipermeable wall. Such polymers are known to the art, as exemplified by U.S. Pat. Nos. 3,845,770, 3,916,899, and 4,160,020, and by the Handbook of Common Polymers, by Scott, J. R. and Roff, W. J., 1971, published by CRC Press, Cleveland, Ohio.

A semipermeable membrane 22 applied to a controlled release dosage form of the present invention may also include a flux regulating agent. The flux regulating agent is a compound added to assist in regulating the fluid permeability or flux through the semipermeable membrane 22. The flux regulating agent can be a flux enhancing agent or a flux decreasing agent and may be preselected to increase or decrease the liquid flux. Agents that produce a marked increase in permeability to fluids such as water are often essentially hydrophilic, while those that produce a marked decrease to fluids such as water are essentially hydrophobic. The amount of regulator in the wall when incorporated therein generally is from about 0.01% to 20% by weight or more. The flux regulating agents in one embodiment include polyhydric alcohols, polyalkylene glycols, polyalkylenediols, polyesters of alkylene glycols, and the like. Typical flux enhancers include the following: polyethylene glycol 300, 400, 600, 1500, 4000, 6000, poly(ethylene glycol-co-propylene glycol); low molecular weight gylcols such as polypropylene glycol, polybutylene glycol and polyamylene glycol; polyalkylenediols, such as poly(1,3-propanediol), poly(1,4-butanediol), poly(1,6-hexanediol); aliphatic diols, such as 1,3-butylene glycol, 1,4-pentamethylene glycol, 1,4-hexamethylene glycol; alkylene triols, such as glycerine, 1,2,3-butanetriol, 1,2,4-hexanetriol, 1,3,6-hexanetriol; and esters such as ethylene glycol dipropionate, ethylene glycol butyrate, butylene glucol dipropionate, and glycerol acetate esters. Representative flux decreasing agents include the following: phthalates substituted with an alkyl or alkoxy or with both an alkyl and alkoxy group, such as diethyl phthalate, dimethoxyethyl phthalate, dimethyl phthalate, and [di(2-ethylhexyl)phthalate]; aryl phthalates, such as triphenyl phthalate, and butyl benzyl phthalate; insoluble salts, such as calcium sulphate, barium sulphate, and calcium phosphate; insoluble oxides, such as titanium oxide; polymers in powder, granule, and like form, such as polystyrene, polymethylmethacrylate, polycarbonate, and polysulfone; esters, such as citric acid esters esterfied with long chain alkyl groups; inert and substantially water impermeable fillers; and resins compatible with cellulose based wall forming materials.

In addition, a semipermeable membrane 22 useful in a controlled release dosage form 10 of the present invention may include materials, such as a plasticizer, which impart flexibility and elongation properties to the semipermeable membrane 22. Exemplary materials that will render the semipermeable membrane 22 less brittle and impart greater tear strength to the semipermeable membrane 22, include phthalate plasticizers, such as dibenzyl phthalate, dihexyl phthalate, butyl octyl phthalate, straight chain phthalates of six to eleven carbons, di-isononyl phthalte, and di-isodecyl phthalate. Suitable plasticizers further include, for example, nonphthalates, such as triacetin, dioctyl azelate, epoxidized tallate, tri-isoctyl trimellitate, tri-isononyl trimellitate, sucrose acetate isobutyrate, and epoxidized soybean oil. Where incorporated in a semipermeable membrane 22, a plasticizer will generally account for about 0.01 wt % to about 20 wt %, or higher, of the membrane formulation.

The expression “exit orifice” as used herein comprises means and methods suitable for releasing the formulation 14 contained within a controlled release dosage form 10 of the present invention. An exit orifice 24 included in a controlled release dosage form 10 according to the present invention may include a passageway, aperture, hole, bore, pore, and the like through the semipermeable membrane 22, or through the semipermeable membrane 22 and the wall 13 of the capsule 12 used to form the controlled release dosage form 10. Alternatively, the exit orifice 24 may include, for example, a porous element, porous overlay, porous insert, hollow fiber, capillary tube, microporous insert, or microporous overlay. The exit orifice 24 can be formed by mechanical drilling or laser drilling, by eroding an erodible element, such as a gelatin plug or a pressed glucose plug, or by crimping the walls to yield the exit orifice 24 when the dosage form is in the environment of use. In an embodiment, the exit orifice 24 in wall 13 is formed in the environment of use in response to the hydrostatic pressure generated within the controlled release dosage form 10. If desired or necessary, the controlled release dosage form 10 can be manufactured with two or more exit orifices (not shown) for delivering formulation 14 during use. A detailed description of orifices and exemplary maximum and minimum dimensions of exit orifices used in controlled release dosage form are disclosed in U.S. Pat. Nos. 3,845,770, 3,916,899, and 4,200,098, the contents of which are herein incorporated by this reference.

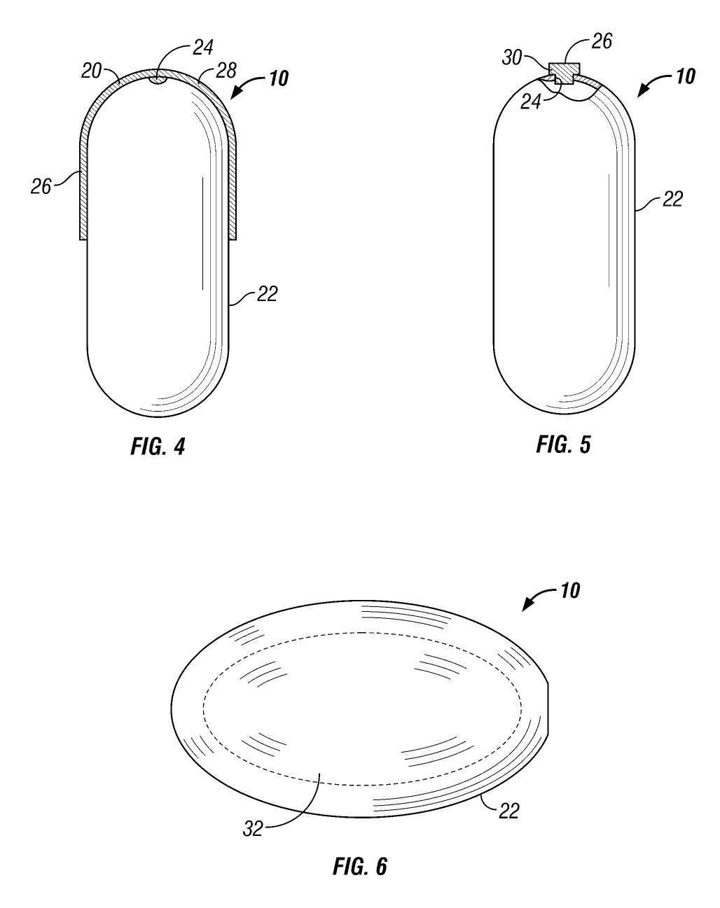

If included in a controlled release dosage form 10 of the present invention, a closure 26 sealing the exit orifice 24 may be provided by any one of several means. For instance, as illustrated in FIG. 4, the closure 26 may simply include a layer 28 of material that covers the exit orifice 24 and is arranged over a portion of the lead end 20 of the dosage form. Alternatively, as shown in FIG. 5, closure 26 may include a stopper 30, such as a bung, cork, or impermeable plug, formed or positioned within the exit orifice 24. Regardless of its specific form, the closure 26 comprises a material impermeable to the passage of fluid, such as high density fluid impermeable polyolefin aluminized polyethylene, rubber, silicon, nylon, synthetic fluorine Teflon®, chlorinated hydrocarbon polyolefins, and fluorinated vinyl polymers. Further, where included, the closure 26 may formed in any suitable shape using any suitable manufacturing technique.

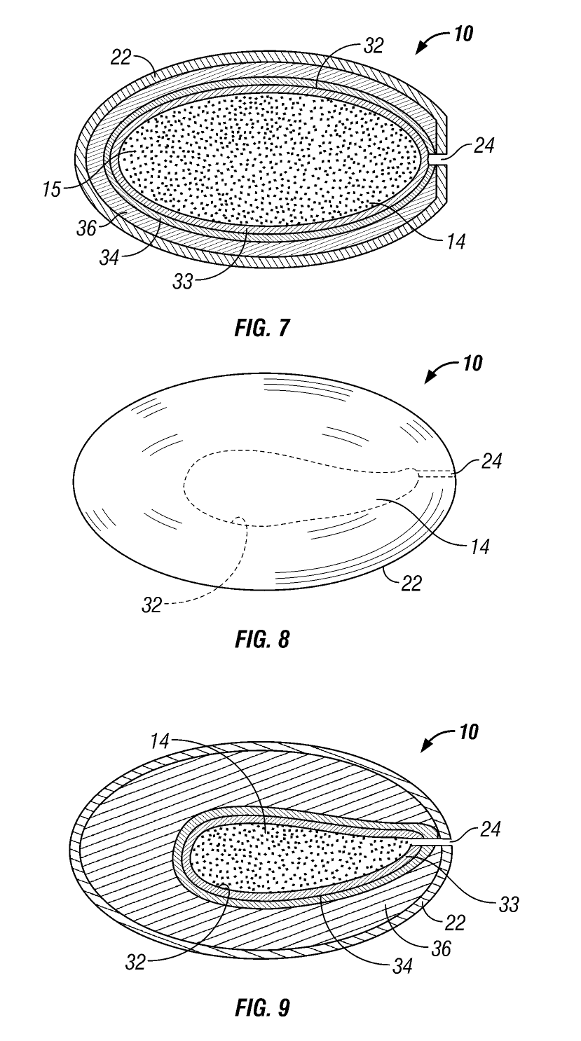

The controlled release dosage form of the present invention may also be formed using a soft gelatin capsule (soft-cap), shown in FIG. 6-FIG. 19. Where a soft-cap is used to form the controlled release dosage form 10 of the present invention, the dosage form 10 includes a soft-cap 32 containing a formulation 14 of the present invention including a hydrophilic macromolecule 15. A barrier layer 34 is formed around the soft-cap 32, and an osmotic layer 36 is formed around the barrier layer 34. Like the hard-cap controlled release dosage form already described, a soft-cap controlled release dosage form 10 according to the present invention is also provided with a semipermeable membrane 22, the semipermeable membrane 22 being formed over the osmotic layer 36. In addition, a soft-cap controlled release dosage form 10 according to the present invention will generally include an enteric coating (not illustrated) as already described. An exit orifice 24 is preferably formed through the semipermeable membrane 22, the osmotic layer 36, and the barrier layer 34 to facilitate delivery of the formulation 14 from the soft-cap controlled release dosage form 10.

The soft-cap 32 used to create a controlled release dosage form 10 of the present invention may be a conventional gelatin capsule, and may be formed in two sections or as a single unit capsule in its final manufacture. Preferably, due to the presence of the barrier layer 34, the wall 33 of the soft-cap 32 retains its integrity and gel-like characteristics, except where the wall 33 dissolves in the area exposed at the exit orifice 24. Generally maintaining the integrity of the wall 33 of the soft-cap 32 facilitates well-controlled delivery of the formulation 14. However, some dissolution of portions of the soft-cap 32 extending from the exit orifice 24 during delivery of the formulation 14 may be accommodated without significant impact on the release rate or release rate profile of the formulation 14.

Any suitable soft-cap may be used to form a controlled release dosage form according to the present invention. The soft-cap 32 may be manufactured in accordance with conventional methods as a single body unit comprising a standard capsule shape. Such a single-body soft-cap typically may be provided in sizes from 3 to 22 minims (1 minimim being equal to 0.0616 ml) and in shapes of oval, oblong, or others. The soft cap 32 may also be manufactured in accordance with conventional methods as a two-piece hard gelatin capsule that softens during operation, such as by hydration. Such capsules are typically manufactured in standard shapes and various standard sizes, conventionally designated as (000), (00), (0), (1), (2), (3), (4), and (5), with largest number corresponding to the smallest capsule size. However, whether the soft-cap 32 is manufactured using soft gelatin capsule or hard gelatin capsule that softens during operation, the soft-cap 32 may be formed in non-conventional shapes and sizes if required or desired for a particular application.

At least during operation, the wall 33 of the soft-cap 32 should be soft and deformable to achieve a desired release rate or release rate profile. The wall 33 of a soft-cap 32 used to create a controlled release dosage form 10 according to the present invention will typically have a thickness that is greater than the thickness of the wall 13 of a hard-cap 12 used to create a hard-cap controlled release dosage form 10. For example, soft-caps may have a wall thickness on the order of 10-40 mils, with about 20 mils being typical, whereas hard-caps may have a wall thickness on the order of 2-6 mils, with about 4 mils being typical. U.S. Pat. No. 5,324,280 describes the manufacture of various soft-caps useful for the creation of controlled release dosage form according to the present invention, and the contents of U.S. Pat. No. 5,324,280 are herein incorporated by this reference.

The barrier layer 34 formed around the soft-cap 32 is deformable under the pressure exerted by the osmotic layer 36 and is preferably impermeable (or less permeable) to fluids and materials that may be present in the osmotic layer 36 and in the environment of use during delivery of the formulation 14 contained within the soft-cap 32. The barrier layer 34 is also preferably impermeable (or less permeable) to the formulation 14 of the present invention. However, a certain degree of permeability of the barrier layer 34 may be permitted if the release rate or release rate profile of the formulation 14 is not detrimentally affected. As it is deformable under forces applied by osmotic layer 36, the barrier layer 34 permits compression of the soft-cap 32 as the osmotic layer 36 expands. This compression, in turn, forces the formulation 14 from the exit orifice 24. Preferably, the barrier layer 34 is deformable to such an extent that the barrier layer 34 creates a seal between the osmotic layer 36 and the semipermeable layer 22 in the area where the exit orifice 24 is formed. In that manner, barrier layer 34 will deform or flow to a limited extent to seal the initially exposed areas of the osmotic layer 36 and the semipermeable membrane 22 when the exit orifice 24 is being formed.

Suitable materials for forming the barrier layer 34 include, for example, polyethylene, polystyrene, ethylene-vinyl acetate copolymers, polycaprolactone and Hytrel® polyester elastomers (Du Pont), cellulose acetate, cellulose acetate pseudolatex (such as described in U.S. Pat. No. 5,024,842), cellulose acetate propionate, cellulose acetate butyrate, ethyl cellulose, ethyl cellulose pseudolatex (such as Surelease® as supplied by Colorcon, West Point, Pa. or Aquacoat™ as supplied by FMC Corporation, Philadelphia, Pa.), nitrocellulose, polylactic acid, poly-glycolic acid, polylactide glycolide copolymers, collagen, polyvinyl alcohol, polyvinyl acetate, polyethylene vinylacetate, polyethylene teraphthalate, polybutadiene styrene, polyisobutylene, polyisobutylene isoprene copolymer, polyvinyl chloride, polyvinylidene chloride-vinyl chloride copolymer, copolymers of acrylic acid and methacrylic acid esters, copolymers of methylmethacrylate and ethylacrylate, latex of acrylate esters (such as Eudragit® supplied by RohmPharma, Darmstaat, Germany), polypropylene, copolymers of propylene oxide and ethylene oxide, propylene oxide ethylene oxide block copolymers, ethylenevinyl alcohol copolymer, polysulfone, ethylene vinylalcohol copolymer, polyxylylenes, polyalkoxysilanes, polydimethyl siloxane, polyethylene glycol-silicone elastomers, electromagnetic irradiation crosslinked acrylics, silicones, or polyesters, thermally crosslinked acrylics, silicones, or polyesters, butadiene-styrene rubber, and blends of the above.

Preferred materials for the formation of the barrier layer 34 include, for example, cellulose acetate, copolymers of acrylic acid and methacrylic acid esters, copolymers of methylmethacrylate and ethylacrylate, and latex of acrylate esters. Preferred copolymers include the following: poly(butyl methacrylate), (2-dimethylaminoethyl)methacrylate, methyl methacrylate) 1:2:1, 150,000, sold under the trademark EUDRAGIT E; poly(ethyl acrylate, methyl methacrylate) 2:1, 800,000, sold under the trademark EUDRAGIT NE 30 D; poly (methacrylic acid, methyl methacrylate) 1:1, 135,000, sold under the trademark EUDRAGIT L; poly(methacrylic acid, ethyl acrylate) 1:1, 250,000, sold under the trademark EUDRAGIT L; poly(methacrylic acid, methyl methacrylate) 1:2, 135,000, sold under the trademark EUDRAGIT S; poly(ethyl acrylate, methyl methacrylate, trimethylammonioethyl methacrylate chloride) 1:2:0.2, 150,000, sold under the trademark EUDRAGIT RL; and poly(ethyl acrylate, methyl methacrylate, trimethylammonioethyl methacrylate chloride) 1:2:0.1, 150,000, sold as EUDRAGIT RS. In each case, the ratio x:y:z indicates the molar proportions of the monomer units and the last number is the number average molecular weight of the polymer. Especially preferred are cellulose acetate containing plasticizers such as acetyl tributyl citrate and ethylacrylate methylmethylacrylate copolymers such as Eudragit NE.

Where desired, a plasticizer may be compounded with the material used to fabricate the soft-cap 32 or the barrier layer 34. Inclusion of a plasticizer increases the flow prospects of the material and enhances the workability of the material during manufacture of the soft cap 32 or the barrier layer 34. For example, glycerin can be used for plasticizing gelatin, pectin, casein or polyvinyl alcohol. Other plasticizers that can be used for the present purpose include, for example, triethyl citrate, diethyl phthalate, diethyl sebacate, polyhydric alcohols, triacetin, polyethylene glycol, glycerol, propylene glycol, acetate esters, glycerol triacetate, triethyl citrate, acetyl triethyl citrate, glycerides, acetylated monoglycerides, oils, mineral oil, castor oil and the like. Where included, the amount of plasticizer in a formulation used to create a soft-cap 32 will generally range from about 0.05 wt % to about 30 wt %, while the amount of plasticizer in a formulation used to create a barrier layer 34 may be as high as about 10 wt % to about 50 wt %.

The osmotic layer 36 included in a soft-cap controlled release dosage form 10 according to the present invention includes a hydro-activated composition that expands in the presence of water, such as that present in gastric fluids. The osmotic layer 36 may be prepared using materials such as those already described in relation to the hard-cap controlled release dosage form previously described. As the osmotic layer 36 imbibes and/or absorbs external fluid, it expands and applies a pressure against the barrier layer 34 and the wall 33 of the gel-cap 32, thereby forcing the formulation 14 through the exit orifice 24.

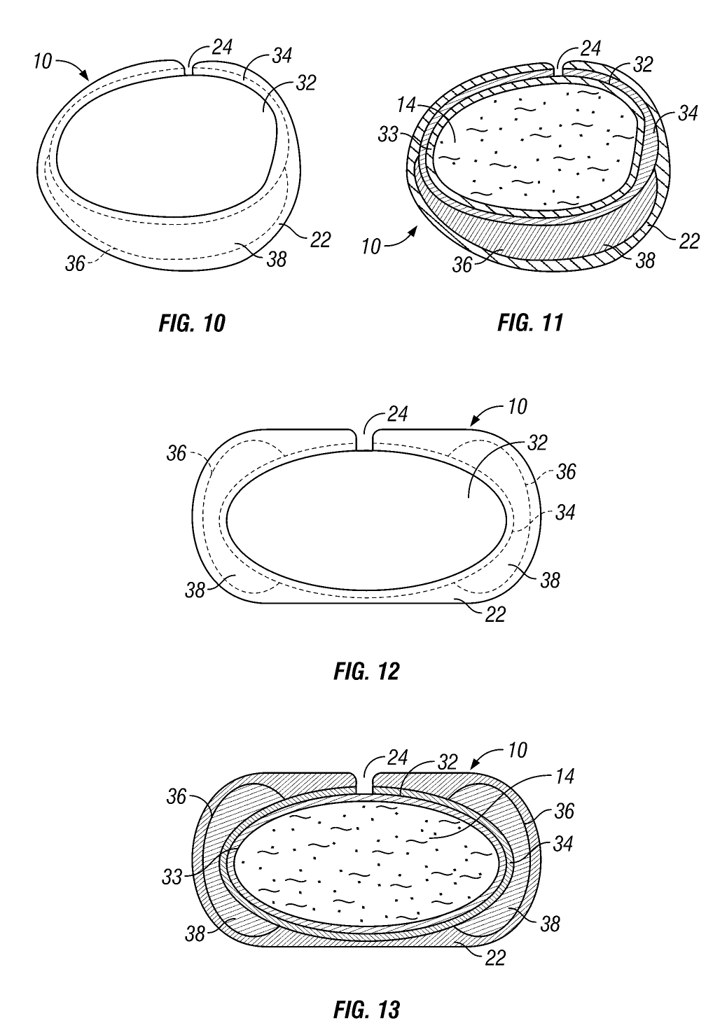

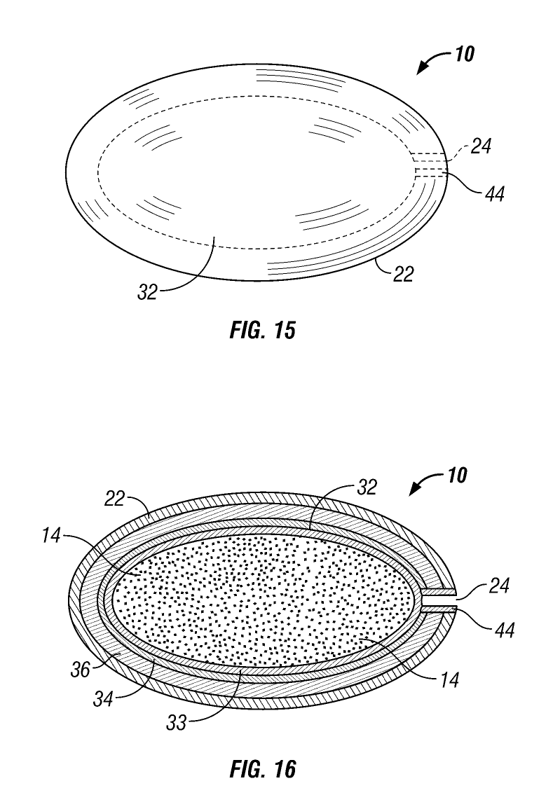

As shown in FIG. 6, FIG. 10-FIG. 13, and FIG. 15-FIG. 16, the osmotic layer 36 included in a soft-cap controlled release dosage form 10 of the present invention may be configured as desired to achieve a desired release rate or release rate profiles, as well as a desired delivery efficiency. For example, the osmotic layer 36 may be an unsymmetrical hydro-activated layer (shown in FIG. 10 and FIG. 11), having a thicker portion remote from the exit orifice 24. The presence of the unsymmetrical hydro-activated layer functions to assure that the maximum dose of formulation 14 is delivered from the dosage form 10, as the thicker section of the osmotic layer 36 swells and moves towards the exit orifice 24. As is easily appreciated by reference to the figures, the osmotic layer 36 may be formed in one or more discrete sections 38 that do not entirely encompass the barrier layer 34 formed around the soft cap 32 (shown in FIG. 10-FIG. 13). As can be seen from FIG. 10 and FIG. 11, the osmotic layer 36 may be a single element 40 that is formed to fit the shape of the soft-cap 32 at the area of contact. Alternatively, the osmotic layer 36 may include two or more discrete sections 38 formed to fit the shape of the soft-cap 32 in the areas of contact (shown in FIG. 12 and FIG. 13).

The osmotic layer 36 may be fabricated using know materials and know fabrication techniques. For example, the osmotic layer maybe fabricated conveniently by tableting to form an osmotic layer 36 of a desired shape and size. For example, the osmotic layer 36 may be tableted in the form a of a concave surface that is complementary to the external surface of the barrier layer 34 formed on the soft-cap 32. Appropriate tooling such as a convex punch in a conventional tableting press can provide the necessary complementary shape for the osmotic layer. Where it is formed by tableting, the osmotic layer 36 is granulated and compressed, rather than formed as a coating. Methods of forming an osmotic layer by tableting are described, for example, in U.S. Pat. Nos. 4,915,949, 5,126,142, 5,660,861, 5,633,011, 5,190,765, 5,252,338, 5,620,705, 4,931,285, 5,006,346, 5,024,842, and 5,160,743, the contents of which are incorporated herein by this reference.

The semipermeable membrane 22 formed around the osmotic layer 36 is non-toxic and maintains its physical and chemical integrity during operation of the soft-cap controlled release dosage form 10. The semipermeable membrane 22 is created using a comprising a composition that does not adversely affect the subject or the other components of the soft-cap controlled release dosage form 10. The semipermeable membrane 22 is permeable to the passage of fluid such as water and biological fluids, but it is substantially impermeable to the passage of the formulation 14 contained within the soft-cap 32 and of the materials forming the osmotic layer 36. For ease of manufacture, it is preferred that the whole of the layer formed around the osmotic layer 36 be a semipermeable membrane 22. The semipermeable compositions used for forming the semipermeable membrane 22 are essentially non-erodible, and they are insoluble in biological fluids during the operational lifetime of the osmotic system. Those materials already set forth as suitable for forming the semipermeable membrane 22 of the previously described hard-cap controlled release dosage form 10 are also suitable for forming the semipermeable membrane 22 of a soft-cap controlled release dosage form 10. The release rate or release rate profile of a soft-cap controlled release dosage form 10 can be controlled by adjusting the thickness or chemical make-up of the semipermeable membrane 22.

The barrier layer 34, osmotic layer 36, and semipermeable layer 22 may be applied to the exterior surface of the soft-cap 32 by conventional coating procedures. For example, conventional molding, forming, spraying, or dipping processes may be used to coat the soft-cap with each layer forming composition. An air suspension procedure that may be used to coat one or more layers on a controlled release dosage form of the present invention is described in U.S. Pat. No. 2,799,241; J. Am. Pharm. Assoc., Vol. 48, pp. 451-59, 1979; and ibid, Vol. 49, pp. 82-84, 1960. Other standard manufacturing procedures are described in Modern Plastic Encyclopedia, Vol. 46, pp. 62-70, 1969; and in Pharmaceutical Sciences, by Remington, 18th Ed., Chapter 90, 1990, published by Mack Publishing Co., Easton, Pa.

Exemplary solvents suitable for manufacturing the various layers of the controlled release soft-cap dosage form 10 of the present invention include inert inorganic and organic solvents that do not adversely harm the materials, the soft-cap, or the final laminated composite structure. The solvents broadly include, for example, members selected from the group consisting of aqueous solvents, alcohols, ketones, esters, ethers, aliphatic hydrocarbons, halogenated solvents, cycloaliphatic, aromatics, heterocyclic solvents and mixtures thereof. Specific solvents that may be used to manufacture the various layers of the soft-cap controlled release dosage form 10 of the present invention include, for example, acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride, nitroethane, nitropropane, tetrachloroethane, ethyl ether, isopropyl ether, cyclohexane, cyclooctane, benzene, toluene, naphtha, 1,4-dioxane, tetrahydrofuran, diglyme, water, aqueous solvents containing inorganic salts, such as sodium and acetone and water, acetone and methanol, acetone and ethyl alcohol, methylene dichloride and methanol, and ethylene dichloride and methanol.

In a preferred embodiment, the exit orifice 24 of a soft-cap controlled release dosage form 10 of the present invention will extend only through the semipermeable layer 22, the osmotic layer 36, and the barrier layer 34 to the wall 33 of the soft cap 32. However, the exit orifice 24 may extend partially into the wall 33 of soft cap 32, as long as the exit orifice 24 does not completely traverse the wall 33. When exposed to the environment of use, the fluids in the environment of use may dissolve the wall 33 of the soft-cap 32 where the soft-cap 32 is exposed at the exit orifice 24, or the pressure exerted on the soft-cap 32 and the barrier layer 34 by the osmotic layer 36 may cause the wall 33 of the gel-cap 32 to rupture where it is exposed to the exit orifice 24. In either case, the interior of the gel-cap 32 will be placed in fluid communication with the environment of use, and the formulation 14 will be dispensed through exit orifice 24 as the barrier layer 34 and the soft-cap 32 are compressed.

The exit orifice 24 formed in the soft-cap controlled release dosage form 10 can be formed by mechanical drilling, laser drilling, eroding an erodible element, extracting, dissolving, bursting, or leaching a passageway former from the composite wall. The passageway can be a pore formed by leaching sorbitol, lactose or the like from a wall or layer as disclosed in U.S. Pat. No. 4,200,098. This patent discloses pores of controlled-size porosity formed by dissolving, extracting, or leaching a material from a wall, such as sorbitol from cellulose acetate. A preferred form of laser drilling is the use of a pulsed laser that incrementally removes material to the desired depth to form the exit orifice 24.

It is presently preferred that a soft-cap controlled-release dosage form 10 of the present invention include mechanism for sealing any portions of the osmotic layer 36 exposed at the exit orifice 24. Such a sealing mechanism prevents the osmotic layer 36 from leaching out of the system during delivery of formulation 14. In one embodiment, the exit orifice 24 is drilled and the exposed portion of the osmotic layer 36 is sealed by barrier layer 34, which, because of its rubbery, elastic-like characteristics, flows outwardly about the inner surface of exit orifice 24 during and/or after the formation of the exit orifice 24. In that manner, the barrier layer 34 effectively seals the area between the osmotic layer 34 and semipermeable layer 22. This can be seen most clearly in FIG. 9. In order to flow and seal, the barrier layer 34 should have a flowable, rubbery-like consistency at the temperature at which the system operation takes place. Materials, such as copolymers of ethyl acrylate and methyl methacrylate, especially Eudragit NE 30D supplied by RohmPharma, Darmstaat, Germany, are preferred. A soft-cap controlled release dosage form 10 having such a sealing mechanisms may be prepared by sequentially coating the soft-cap 32 with a barrier layer 34, an osmotic layer 36, and semipermeable layer 22 and then drilling the exit orifice 24 to complete the dosage form 10.

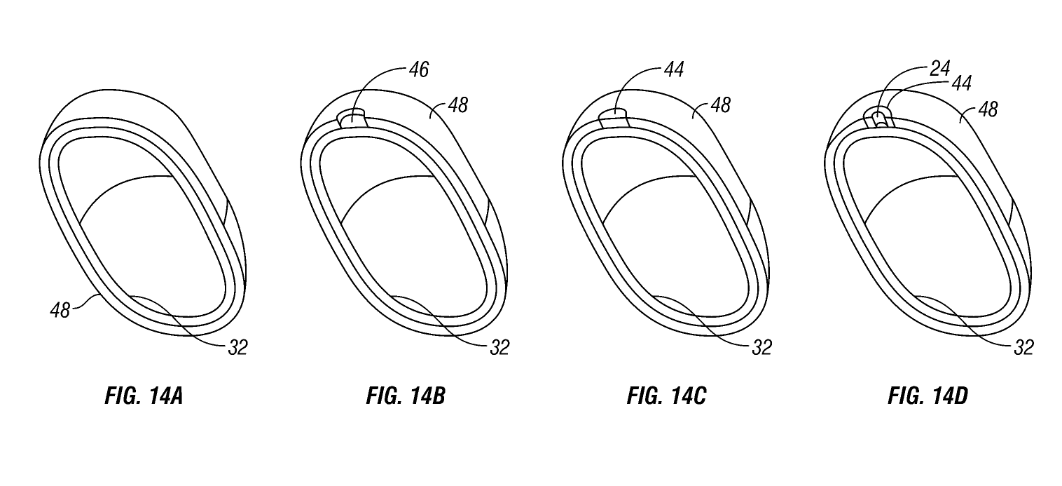

Alternatively a plug 44 may be used to form the desired sealing mechanism for the exposed portions of the osmotic layer 36. As is shown in FIG. 14A through FIG. 14D, a plug 44 may be formed by providing a hole 46 in the semipermeable membrane and the barrier layer (shown as a single composite membrane 48). The plug 44 is then formed by filling the hole 46 with, for example, a liquid polymer that can be cured by heat, radiation or the like (shown in FIG. 14C). Suitable polymers include polycarbonate bonding adhesives and the like, such as, for example, Loctite® 3201, Loctite® 3211, Loctite® 3321 and Loctite®3301, sold by the Loctite Corporation, Hartford, Conn. The exit orifice 24 is drilled into plug to expose a portion of the soft-cap 32. A completed dosage form having a plug-type seal is illustrated in an overall view of FIG. 15 and in cross-section in FIG. 16.

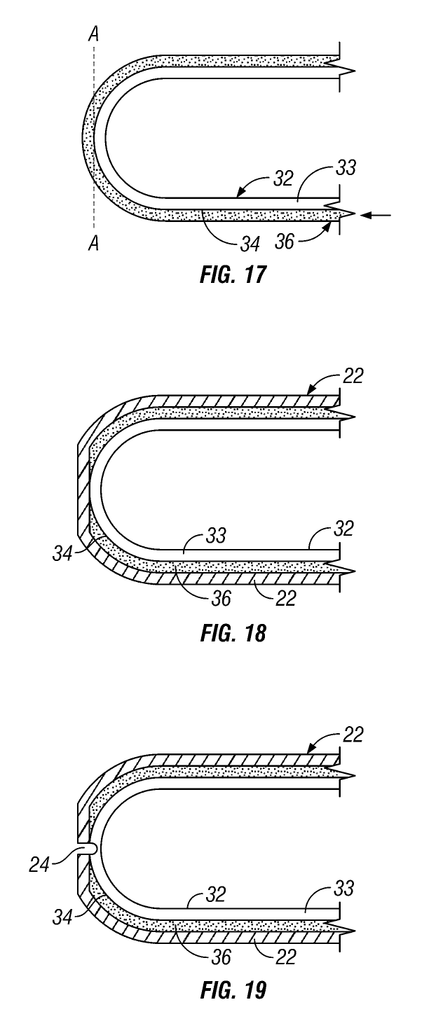

Still another manner of preparing a dosage form having a seal formed on the inner surface of the exit orifice is described with reference to FIG. 17-FIG. 19. In FIG. 17, a soft-cap 32 (only partially shown) has been coated with the barrier layer 34 and an osmotic layer 36. Prior to coating the semipermeable membrane 22, a section of the osmotic layer 36 extending down to, but not through, the barrier layer 34 is removed along line A-A. Then a semipermeable membrane 22 is coated onto the dosage form 10 to yield a precursor of the dosage form such as illustrated in FIG. 18. As can be seen from FIG. 18, the portion of gel-cap 32 where the exit orifice 24 is to be formed is covered by the semipermeable membrane 22 and the barrier layer 34, but not the osmotic layer 36. Consequently, when an exit orifice 24 is formed in that portion of the dosage form 10, as can be seen most clearly in FIG. 19, the barrier layer 34 forms a seal at the juncture of the semipermeable membrane 22 and expandable layer 20 such that fluids may pass to osmotic layer 36 only through the semipermeable membrane 22. Accordingly, osmotic layer 36 is not leached out of the dosage form 10 during operation. The sealing aspect of the soft-cap controlled release dosage form 10 of the present invention allows the rate of flow of fluids to the osmotic layer 36 to be carefully controlled by controlling the fluid flow characteristics of the semipermeable membrane 22.

The various layers forming the barrier layer, expandable layer (when not a tableted composition) and semipermeable layer may be applied by conventional coating methods such as described in U.S. Pat. No. 5,324,280, previously incorporated herein by reference. While the barrier layer, expandable layer and semipermeable layer forming the multilayer wall superposed on the soft-cap have been illustrated and described for convenience as single layers, each of those layers may be composites of several layers. For example, for particular applications it may be desirable to coat the soft-cap with a first layer of material that facilitates coating of a second layer having the permeability characteristics of the barrier layer. In that instance, the first and second layers comprise the barrier layer as used herein. Similar considerations would apply to the semipermeable layer and the expandable layer.

In the embodiment shown in FIG. 10 and FIG. 11, the barrier layer 34 is first coated onto the gelatin capsule 12 and then the tableted, osmotic layer 36 is attached to the barrier-coated soft-cap with a biologically compatible adhesive. Suitable adhesives include, for example, starch paste, aqueous gelatin solution, aqueous gelatin/glycerin solution, acrylate-vinylacetate based adhesives such as Duro-Tak adhesives (National Starch and Chemical Company), aqueous solutions of water soluble hydrophilic polymers such as hydroxypropyl methyl cellulose, hydroxymethyl cellulose, hydroxyethyl cellulose, and the like. That intermediate dosage form is then coated with a semipermeable membrane. The exit orifice 24 is formed in the side or end of the soft-cap 32 opposite the osmotic layer 36. As the osmotic layer 36 imbibes fluid, it will swell. Since it is constrained by the semipermeable membrane 22, the osmotic layer 36 compresses the soft-cap 32 as the osmotic layer 36 expands, thereby expressing the formulation 14 from the interior of the soft-cap 32 into the environment of use.

As mentioned, the soft-cap controlled release dosage form 10 of the present invention may include an osmotic layer formed of a plurality of discrete sections. Any desired number of discrete sections may be used, but typically the number of discrete sections will range from 2 to 6. For example, two sections 38 may be fitted over the ends of the barrier-coated soft-cap 32 as illustrated in FIG. 12 and FIG. 13. FIG. 12 is a schematic of a soft-cap controlled release dosage form 10 with the various components of the dosage form indicated by dashed lines and the soft-cap 32 indicated by a solid line. FIG. 13 is a cross-sectional view of a completed soft-cap controlled release dosage form 10 having two, discrete expandable sections 38. Each expandable section 38 is conveniently formed by tableting from granules and is adhesively attached to the barrier-coated soft-cap 32, preferably on the ends of the soft-cap 32. Then a semipermeable layer 22 is coated on the intermediate structure and an exit orifice 24 is formed in a side of the dosage form between the expandable sections 38. As the expandable sections 38 expand, the formulation 14 will be expressed from the interior of the soft-cap 32 in a controlled manner to provide controlled-release delivery of the formulation 14.

The hard-cap and soft-cap controlled release dosage forms prepared in accordance with the present invention may be constructed as desired to provide controlled release of the formulation of the present invention at a desired release rate or release rate profile over a desired period of time. Preferably, the dosage forms of the present invention are designed to provide controlled release of the formulation of the present invention over a prolonged period of time. As used herein, the phrase “prolonged period of time” indicates a period of time of two or more hours. Typically for human and veterinary pharmaceutical applications, a desired prolonged period of time may be from 2 hours to 24 hours, more often 4 hours to 12 hours or 6 hours to 10 hours. For many applications it may be preferable to provide dosage forms that only need to be administered once-a-day. Additional controlled release delivery devices that may be used to create a controlled release dosage form of the present invention are described in U.S. Pat. Nos. 4,627,850 and 5,413,572, the contents of which are incorporated herein by this reference.

It is believed that a controlled release dosage form will provide functional advantages not achievable by enteric-coated capsules providing a dose-dumping or bolus release of their contents. Controlling the release of the formulation of the present invention within the GI tract over time facilitates greater control of the plasma concentration of the hydrophilic macromolecule delivered using the formulation of the present invention. Greater control of the plasma concentration of the hydrophilic macromolecule delivered, in turn, eases the task of achieving and maintaining therapeutic levels of hydrophilic macromolecule within the subject and may also ease or eliminate side affects. Moreover, it is believed that, relative to a bolus dose, controlled delivery of the formulation of the present invention will further increase the bioavailability of the hydrophilic macromolecule included in the formulation.

Without being limited to specific mechanism, it is thought that the controlled release of the formulation of the present invention may increase the bioavailability of the hydrophilic macromolecule delivered by providing the formulation increased opportunities reach and adhere to the mucosal membrane of the GI tract. Ideally, the formulation of the dosage form is released at or near the surface of the GI mucosal membrane so that the formulation can easily reach and spread across the surface of the GI mucosal membrane with limited interference from the lumenal contents. If the formulation is released at a location that is relatively remote from the GI mucosal membrane, however, there is a higher likelihood that all or some of the formulation will be prevented from reaching the GI mucosal membrane due to interference from the lumenal contents. Unfortunately, precise placement of the dosage form of the present invention relative to the surface of the GI mucosal membrane over time is not presently feasible, and as the dosage form passes through the GI tract, it may move relatively closer to or farther from the surface of the GI mucosal membrane. If the dosage form releases the formulation of the present invention as a bolus dose, the entire volume of the formulation contained within the dosage form may be released at a location relatively remote from the surface of the GI mucosal membrane. In such a scenario, the entire volume of formulation delivered would be subject to interference by the contents of the GI lumen, and, as a result a relatively small amount of the formulation may actually reach the surface of the GI mucosal membrane. In contrast, however, if the dosage form of the present invention releases the formulation of the present invention at a controlled rate over a period of time, as the dosage form passes through the GI tract, the dosage form will likely approach or abut the surface of the GI mucosal membrane at multiple points during its passage, thereby providing multiple opportunities for the formulation to reach and adhere to the GI mucosal membrane. In addition, a controlled release dosage form will tend to release more formulation in the lower GI tract, such as in the colon, where dilution of the formulation and enzymatic degradation of the hydrophilic macromolecule included in the formulation will be minimized.

### EXAMPLE 1

To better appreciate the behavior of the carrier included in the formulation of the present invention, the rheological properties of an exemplary carrier, CREMOPHOR® EL (ethoxylated castor oil), were characterized. To characterize the Theological behavior of CREMOPHOR® EL, the carrier was mixed homogeneously with water in various ratios, and the CREMOPHOR® EL/water blends were measured by a Haak 100 RheoStress Rheometer for η (dynamic viscosity), G′ (storage modulus), G″ (loss modulus), and 6 (G″/G′).

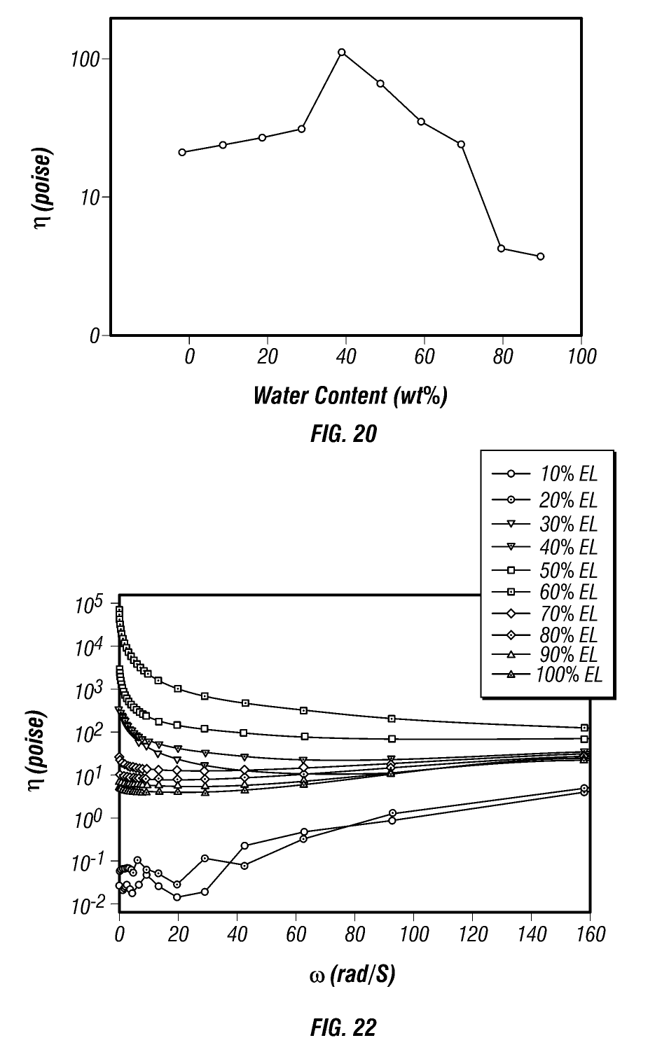

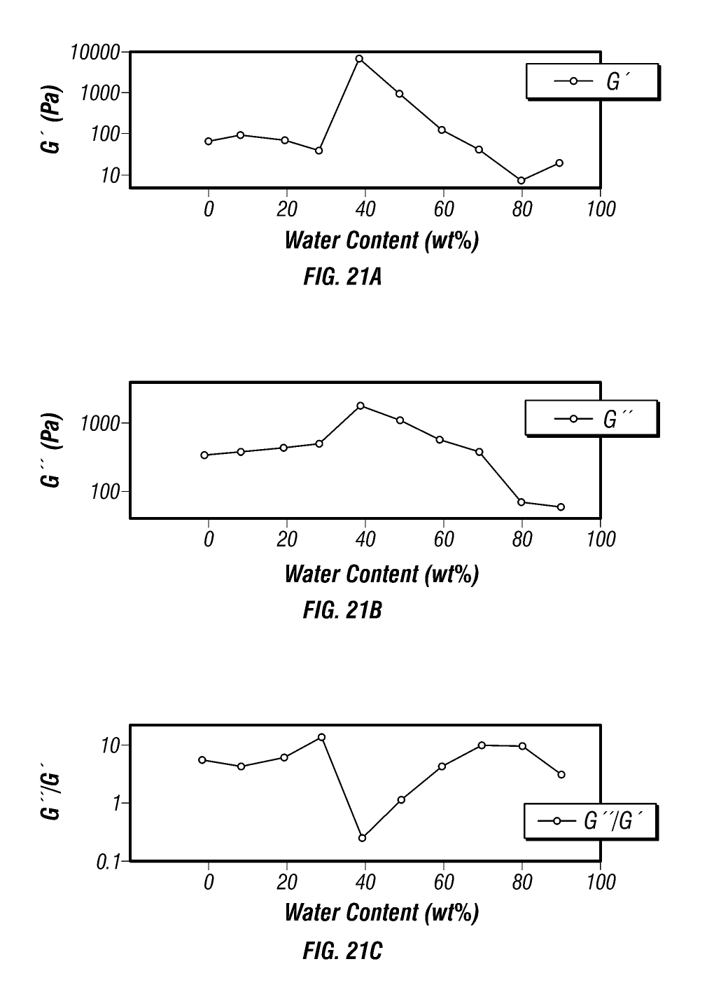

FIG. 20 shows the dynamic viscosity of various CREMOPHOR® EL/water blends as a function of water content. As can be appreciated by reference to FIG. 20, as the water content rose beyond about 30%, the viscosity of the blends increased dramatically, peaking at about 40% water content. However, as the water content continued to increase beyond about 40%, the viscosity of the CREMOPHOR™) EL/water blends began to decrease. As the water content of the CREMOPHOR™) EL/water blends approached 80%, the viscosity of the blends decreased well below the viscosity of CREMOPHOR® EL that is substantially free of water. FIG. 21 shows the G′(storage modulus), G″ (loss modulus), and 6 (G″/G′) of CREMOPHOR® EL/water blends as a function of water content. As the water content of the blends rose, the rheological properties of the blends changed significantly. In particular, as water content rose from about 30% to about 40%, the value of G″/G′transitioned from greater than one (G″/G′>1) to less than one (G″/G′<1), indicating that CREMOPHOR® EL transitions from a liquid-type substance to a rubber-type substance as it absorbs water. However, as the water content of the blends rose beyond 40%, the value of G″/G′ transitioned back from less than one (G″/G′<1) to greater than one (G″/G′>1), which indicates that, as the water content of CREMOPHOR® EL increases beyond about 40%, the material transitions back from a rubber-like substance to a liquid-type substance.

The dynamic viscosity of various CREMOPHOR® EL/water blends were measured at shear rates ranging from 0.0628 rad/s to 628 rad/s. As shown in FIG. 22, shear rate had an inverse effect on the dynamic viscosity of samples containing 30% to 60% CREMOPHOR® EL. It was demonstrated that dynamic viscosity decreased as shear rate increased, which is characteristic of the pseudoelastic behavior of non-Newtonian fluid. Other compositions of CREMOPHOR® EL/water (low viscosity) showed dilatant property (i.e., dynamic viscosity increased as shear rate increased).

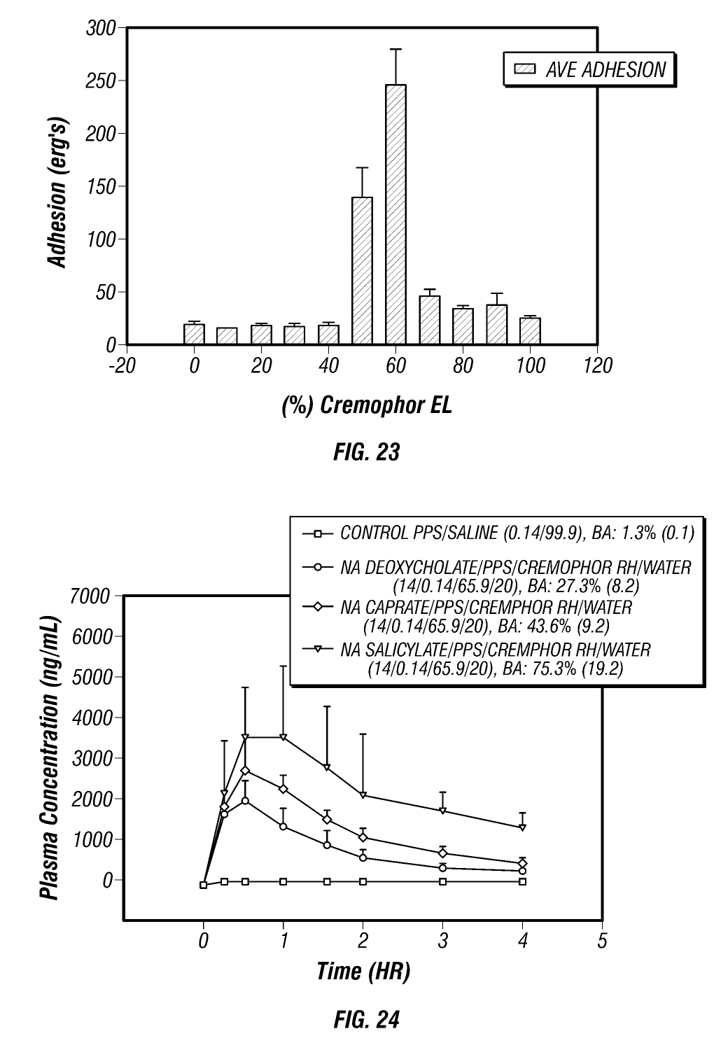

In order to assess the bioadhesive properties of the CREMOPHOR® EL as a function of water content, the adhesion of various CREMOPHOR® EL/water blends to a mucin surface was determined using a texture profile analyzer (TPA) from Texture Technologies Corp. A 500 mg mucin tablet with a flat circular surface area of 0.096 in2 was compressed with a 0.5 ton force. The mucin tablet was firmly attached to the lower end of the TPA probe using double-sided adhesive tape. Samples of CREMOPHOR® EL/water blends of various ratios were prepared in small bottles that were affixed onto the TPA platform. The mucin tablet was moistened in AGF for 60 seconds prior to the measurements. During measurement, the TPA probe with attached mucin tablet was lowered onto the surface of each sample at a constant speed of 1 mm/sec. To ensure the intimate contact between the mucin tablet and the sample, the tabled stayed for 60 seconds before the probe was moved upward. The force required to detach the mucin tabled from the surface of the samples was recorded as a function of time. Adhesion energy (E) was calculated from the AUC of the curve (E=AUC×S). FIG. 23 presents the results of the measurements. The blend of CREMOPHOR® EL/water in the ratio of 60/40 was most adhesive to the surface of the mucin tablet. These results show good correlation between adhesion and viscosity, with the more viscous formulations tending to be the most adhesive as well.

### EXAMPLE 2

The bioavailability of pentosan polysulfate sodium (PPS) administered using various formulations according to the present invention was evaluated. PPS is the active component of Elmiron, a commercial drug indicated for the treatment of interstitial cystitis (IC). The mechanism by which PPS exerts its therapeutic effect remains to be elucidated, but it has been proposed that PPS may provide a therapeutic effect to sufferers of IC by adhering to the mucosal membrane of the urinary bladder and buffering irritating solutes in the urine. Having dense negative charges, PPS is very soluble in water, about 50% by weight, and its molecular weight ranges from 4,000 to 6,000 daltons. The elimination half-life of PPS has a mean value of 24 hours following IV injection. However, the elimination half-life in urine has been determined to be 4.8 hours after oral administration (See, Physicians Desk Reference, page 53, Medical Economics Company, 2001). The oral bioavailability of PPS in humans is very low (approximately 3%), which can be attributed to its hydrophilicity, large molecular size, and dense negative charges. Presently, patients must continue Elmiron therapy for many days in order to achieve an optimal therapeutic plasma level. The low oral bioavailability of PPS not only compromises its efficacy for the treatment of IC, but also limits its applicability for other indications, including glomerulosclerosis, arteriosclerosis, and vascular graft stenosis. Hence, an orally administered formulation that improves oral bioavailability and reduces the time required to achieve clinically therapeutic plasma levels could improve the efficacy with which IC is treated with PPS, reduce the side effects resulting from PPS therapies, and expand the therapeutic indications for PPS.

Evaluation of PPS Bioavailability Using Rat Ileal Models

PPS formulations according to the present invention were first tested using two rat ileal models. Both models utilized male and/or female Sprague Dawley from Charles River rats weighing between 200 g and 450 g, and both models were intracolonic loop models. The first model used was a flushed/ligated (F/L) model, wherein a segment of the ileum is isolated, flushed of lumenal content, and then ligated at both the proximal and distal openings before being dosed with a test formulation. The second model used was a non-flushed/non-ligated (NF/NL) model, wherein a segment of the ileum is isolated and cleared of surrounding omentum, following a midline abdominal incision. The lumenal content of the isolated segment was left undisturbed and a test formulation was injected directly into the lumen of the isolated segment using a needle of suitable gauge (the gauge of the needle varied depending on the viscosity of the test formulation). After dosing with a test formulation, the punctured site was tightly closed with a piece of suture, with the ligation performed parallel to the serosal surface to allow continual flow of lumenal content.

Various tests were conducted using both models. In each test, the formulation(s) used included tritiated PPS, and in each test, blood samples were withdrawn up to four (4) hours after administration. Scintillation counting of plasma samples was performed to assess the PPS concentration in the plasma. Three to four rats were used to evaluate each formulation, and all rats were food fasted overnight and anesthetized intraperitoneally with sodium pentobarbital. In each test conducted using the rat ileal models, the absolute bioavailability of PPS was measured as a percentage of the bioavailability achieved through intravenous administration of PPS.

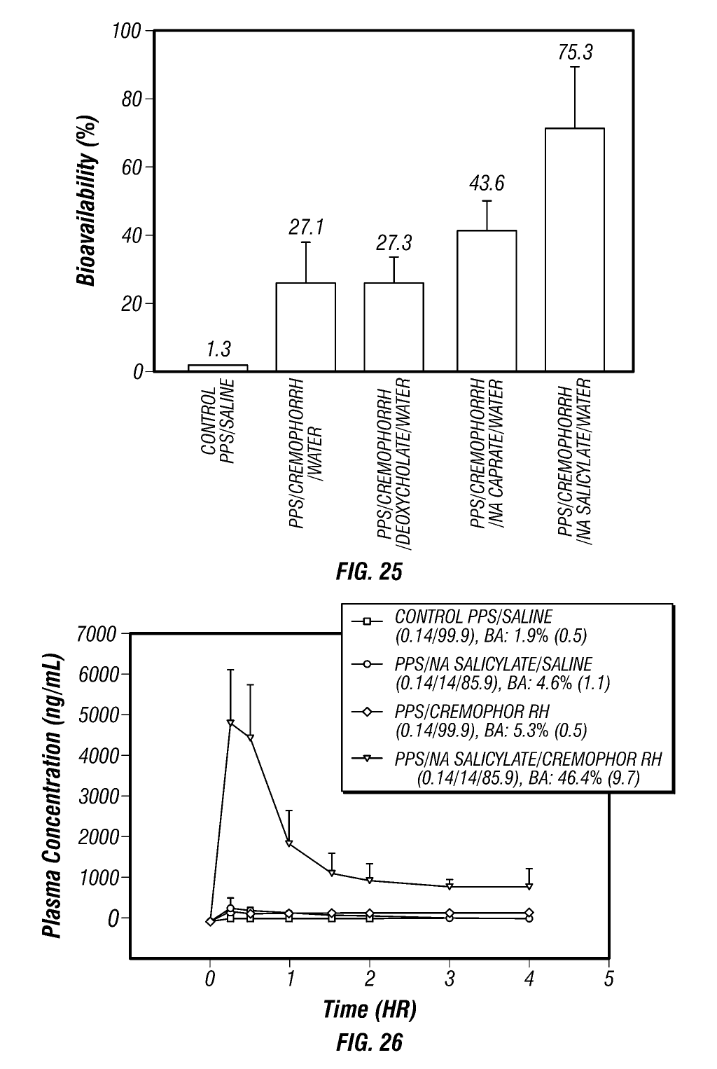

Test formulations containing sodium salicylate, sodium caprate, or sodium deoxycholate as permeation enhancers were tested using the F/L rat model. FIG. 24 and FIG. 25 show the PPS plasma concentration profiles and percent bioavailability achieved with each of the different formulations. The weight percentages (wt %) of each component included in the control formulation and in the test formulations containing sodium deoxycholate, sodium caprate and sodium salicylate, which are represented in FIG. 24 and FIG. 25, are provided in FIG. 24. The formulation of PPS, cremaphor RH, and water, noted in FIG. 25 contained, again in wt %, 0.14% PPS, 79.9% cremaphor RH, and 20% water. The formulation containing sodium salicylate showed the highest bioavailability, with a bioavailability of 75.3%. The formulations containing sodium caprate and sodium deoxycholate yielded bioavailabilities of 43.6% and 27.3%, respectively. In these studies, the PPS was dosed at 1.4 mg/kg body weight, the enhancer was dosed at 140 mg/kg body weight, and the total formulation was dosed at 1 g/kg of body weight.

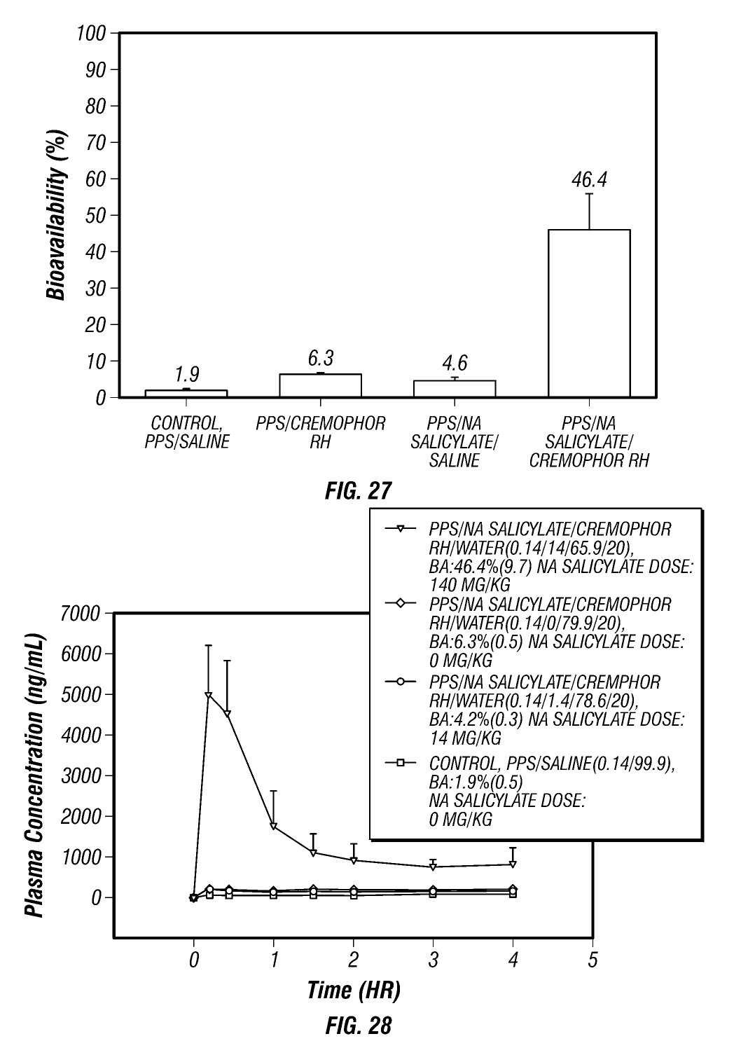

FIG. 26 and FIG. 27 illustrate the PPS plasma concentration profiles and percent bioavailability achieved using four different test formulations administered using the NF/NL model. Both figures emphasize the synergistic effect achieved by administering PPS within a formulation comprising both a permeation enhancer and a carrier capable of forming a bioadhesive gel in-situ. As is easily appreciated by reference to FIG. 26 and FIG. 27, the PPS formulation including a permeation enhancer (sodium salicylate) in saline carrier did not significantly increase the bioavailability of PPS relative to the control. Moreover, the PPS formulation including an in-situ gelling carrier (Cremophor) without a permeation enhancer also failed to significantly increase the bioavailability of PPS relative to the control. However, when a PPS formulation including both a permeation enhancer (sodium salicylate) and an in-situ gelling carrier was administered, the absorption of PPS increased dramatically, yielding a bioavailability of 46.4%. The dose of PPS in each of the four formulations was 1.4 mg/kg, and, where included, the dose of permeation enhancer was 140 mg/kg. Each of the four formulations was dosed at 1 g/kg.

In light of the positive results illustrated in FIG. 26 and FIG. 27, the effect of sodium salicylate dose on PPS absorption was studied using the NF/NL rat model. Three in-situ gelling formulations including three different doses of sodium salicylate (0 mg/kg, 14 mg/kg, and 140 mg/kg) were evaluated. In this study, PPS dose was 1.4 mg/kg and total formulation at 1 g/kg. As expected, when the sodium salicylate dose included in the formulation was 0 mg/kg, the bioavailability of PPS was not significantly enhanced. However, as is shown in FIG. 28, it was surprisingly found that when the sodium salicylate dose was reduced to 14 mg/kg from 140 mg/kg, the formulation also failed to increase PPS bioavailability. It is believed that, in the NF/NL model, a dose of 14 mg/kg of sodium salicylate is ineffective in increasing the bioavailability of PPS because of the dilution of the sodium salicylate by GI lumenal secretions.

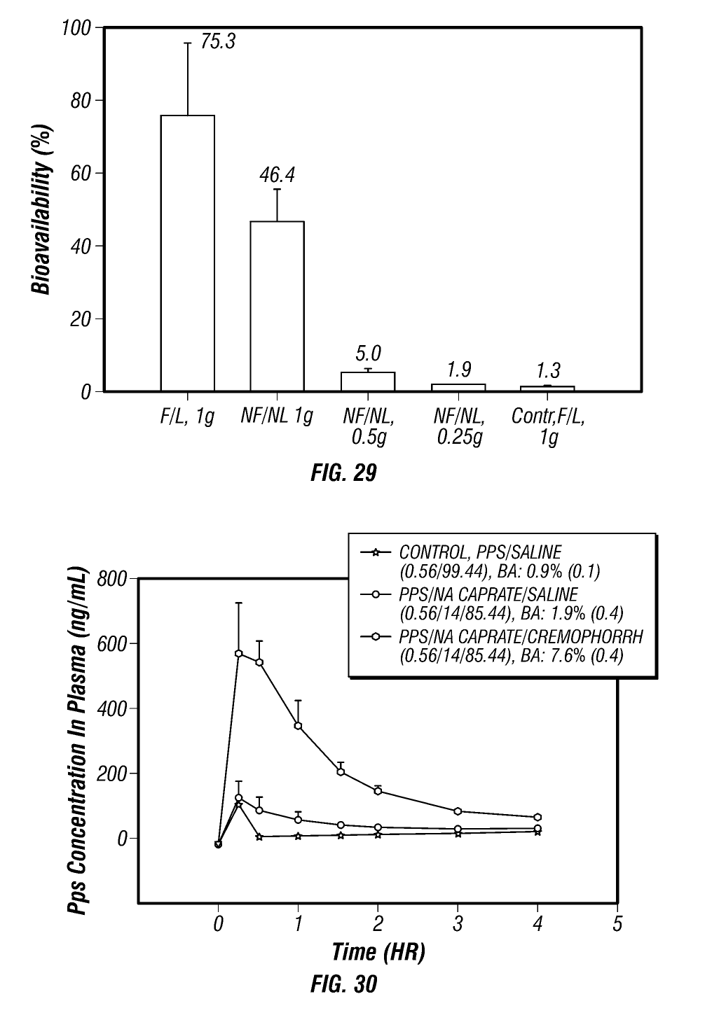

A further rat study was conducted, wherein lower doses of exemplary in-situ gelling formulations were administered using both F/L and the NF/NL ileal models. Four different formulations were prepared for the study, with each formulation providing a PPS dose of 1.4 mg/kg. One of the four formulations was a control formulation containing, by wt %, 0.14% PPS and 99.9% saline. The remaining three formulations administered in the study were in-situ gelling formulations. The first in-situ gelling formulation was administered at a formulation dose of 1.0 g/kg and contained 0.14 wt % PPS, 14 wt % sodium salicylate, 65.9 wt % CREMOPHOR® RH, and 20 wt % water. The second in-situ gelling formulation was administered at a formulation dose of 0.5 g/kg and contained 0.28 wt % PPS, 14 wt % sodium salicylate, 65.72 wt % CREMOPHOR® RH, and 20 wt % water. The third in-situ gelling formulation was administered at a formulation dose of 0.25 g/kg and contained 0.56 wt % PPS, 14 wt % sodium salicylate, 65.44 wt % CREMOPHOR® RH, and 20 wt % water. FIG. 29 summarizes the PPS bioavailability achieved through administration of the different formulations in either a F/L or NF/NL model.

The control formulation was administered in a formulation dose of 1 g/kg in a F/L model and resulted in a PPS bioavailability of 1.3%. The in-situ gelling formulation delivered at a 1 g/kg formulation dose was administered in both a F/L model and a NF/NL model and achieved a PPS bioavailability of 75.3% and 46.4%, respectively. The in-situ gelling formulation delivered at a 0.5 g/kg formulation dose was administered in only a NF/NL model and resulted in a PPS bioavailability of 5.0%. Like the in-situ gelling formulation delivered at a 0.5 g/kg formulation dose, the in-situ gelling formulation delivered at a 0.25 g/kg formulation dose was administered only in a NF/NL model. However, the in-situ gelling formulation delivered at a 0.25/kg formulation dose achieved a PPS bioavailability of only 1.9%. Therefore, the bioavailability of PPS decreased dramatically from 75.3% to 1.9% from the F/L model (at 1 g/kg) to the NF/NL model (at 0.25 g/kg), providing further evidence that, in the NF/NL model, sodium salicylate is diluted by GI lumenal fluid to a concentration below that which is necessary to effectively permeabilize GI enterocytes.

Because the solubility of sodium caprate in water is lower than that of sodium salicylate, a further study was conducted using two test formulations including sodium caprate as a permeation enhancer. Sodium caprate has a lower solubility in water than sodium salicylate. As part of the study, three formulations were evaluated using the NF/NL rat model. Each formulation was dosed at a formulation dose of 0.25 g/kg, and each formulation provided a PPS dose of 1.4 mg/kg. The weight percentages of each constituent of each formulation are indicated in FIG. 30. As can be appreciated by reference to FIG. 30, even at the formulation dose of 0.25 g/kg, the formulation including both sodium caprate and an in-situ gelling carrier (CREMOPHOR® RH) exhibited synergistic effects in enhancing PPS transport across the rat intestinal mucosa. The formulation containing both sodium caprate and CREMOPHOR® RH produced 7.6% BA, compared to the 1.9% bioavailability achieved with sodium caprate alone. Because the solubility of sodium caprate in water is lower than that of sodium salicylate, it is believed that utilization of sodium caprate minimized the dilution effect created in the intestinal lumen.

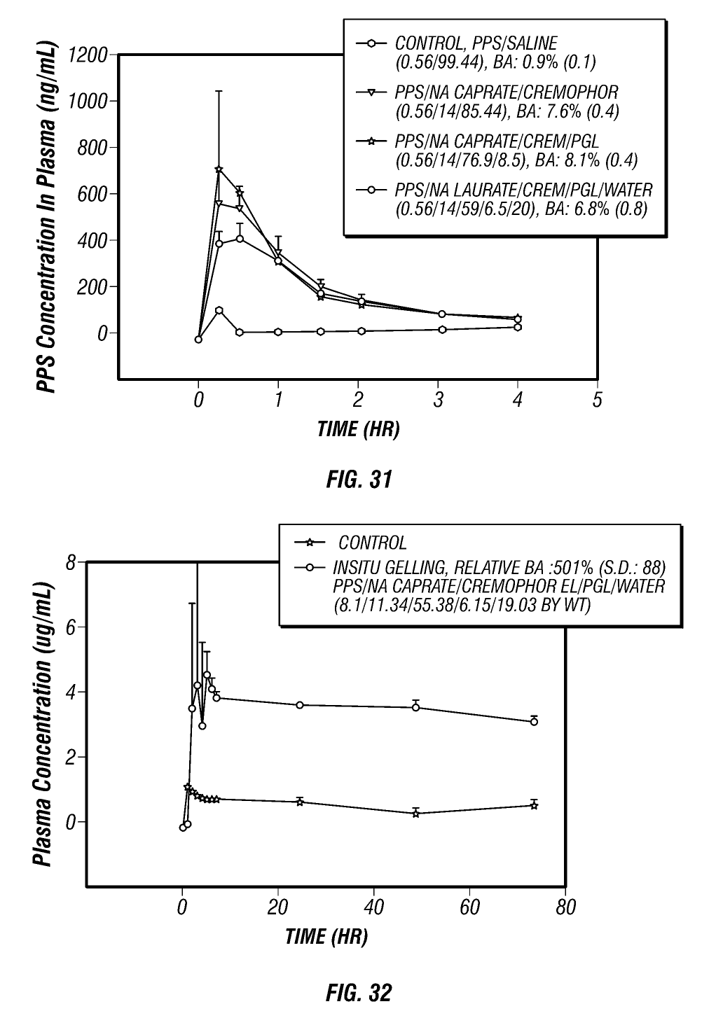

A final rat ileal study was conducted, wherein three test formulations were provided with varying amounts of an exemplary viscosity reducing agent, propylene glycol laurate (PGL). PGL is compatible with Cremophor and with fatty acid type permeation enhancers. The addition of PGL into formulations may help decrease the initial viscosity of an in-situ gelling formulation such that the formulation can more easily spread out across intestinal mucosa before gelling. Each of the three formulations were tested in the NF/NL model, with the first formulation containing 0 wt % PGL, the second formulation containing 8.5 wt % PGL, and the third formulation containing 6.5 wt % PGL. One formulation containing no PGL was tested. The three formulations containing PGL were prepared and tested in the NF/NL rat model. Each formulation was dosed at 0.25 g/kg and each formulation provided a PPS dose of 1.4 mg/kg. The precise composition of each of the three formulations is indicated in FIG. 31.

FIG. 31 shows the PPS plasma concentration vs. time of the three formulations as well as the bioavailability of PPS achieved by each. The formulation including no PGL resulted in a bioavailability of 7.6%. The formulation including 8.5 wt % PGL provided a PPS bioavailability of 8.1%, and the formulation including 6.5 wt % provided a PPS bioavailability of 6.8%.

Evaluation PPS Oral Bioavailability in Dogs

After thorough testing with the rat in-vivo models, a PPS formulation according to the present invention was tested in three beagles. In order to target the formulation to the small intestine (ileum) of the dogs, the in-situ gelling formulation was incorporated into an enteric-coated gelatin capsule. Enteric-coated capsules containing a 100 mg dose of tritiated PPS were made, providing a PPS dose of 15 mg/kg. The formulation included in each capsule contained tritium-labeled PPS/Na caprate/CREMOPHOR® EL/PGL/Water at the following weight percentages: 8.1/11.34/55.38/6.15/19.03. Each dog was fed one capsule using an oral gavage after having been food fasted overnight. After administration of a capsule to each dog, blood samples were drawn from periodically from each dog over a 4-day period, and scintillation counting of plasma samples was performed to assess the PPS concentrations.

As a control, the content of one commercial 100 mg PPS capsule (Elmiron 100 mg) was dissolved in saline, spiked with tritiated PPS, and individually gavaged to each of the same beagles two weeks prior to the administration of the in-situ gelling formulation. After administration of the control formulation, blood samples were again drawn periodically from each dog over a 4-day period, and scintillation counting of plasma samples was performed to assess the PPS concentrations.

The PPS plasma levels from both studies are presented FIG. 32. The in-situ gelling formulation of the present invention provided a Cₘₐₓ of 6.2 μg/ml compared to 1.3 μg/ml for the control. Thus, the relative bioavailability of the PPS orally administered in a formulation according to the present invention was 501%, relative to the PPS bioavailability provided by the control. At tₘₐₓ the in-situ gelling formulation provided a plasma concentration of PPS of 2.5 μg/ml, while the control provided a plasma concentration of PPS of 1.3 μg/ml.

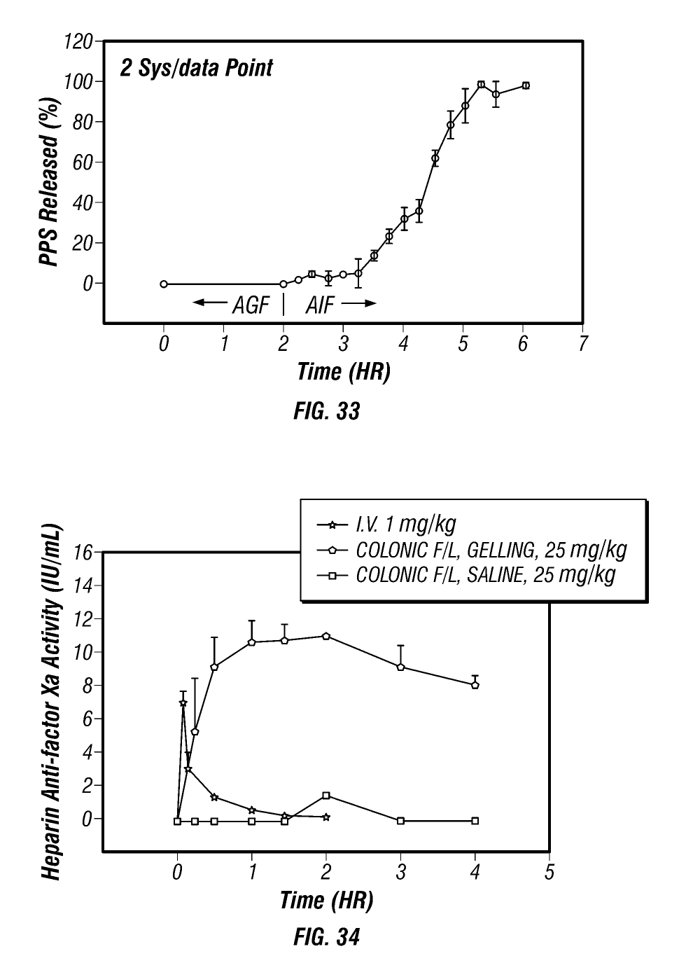

Prior to administering the enteric-coated capsules containing the in-situ gelling formulation to the three beagles, the same in-situ gelling formulation was filled into a “00” enteric coated gelatin capsule and tested in USP dissolution apparatus. In artificial gastric fluid (AGF) or pH 1.2 bathing medium, the filled enteric-coated capsule remained intact, and less than 2% PPS was detected after more than 8 hours of incubation. In a separate test, the enteric-coated capsules were filled with an in-situ gelling formulation including PPS/Na caprate/CREMOPHOR® EL/PGL at 10 wt %/14 wt %/68.4 wt %/7.6 wt %, respectively. These capsules were presoaked in AGF for 2 hours then transferred into artificial intestinal fluid (AIF). The capsules dissolved in AIF and released their content as predicted. FIG. 33 shows the in-vitro release profile of the in-situ gelling formulation in AIF.

### EXAMPLE 3

The bioavailability of unfractionated heparin and low molecular weight heparin (LMWH) delivered using formulations according to the present invention was evaluated. Unfractionated heparin and LMWH are heterogeneous mucopolysaccharides called sulphated glucoaminoglycans characterized by an anti-coagulation property. Unfractionated heparin and LMWH are used to prevent post-operative venous thrombo-embolism and post-operative pulmonary embolism. Both agents are also used to prevent clotting during extracorporeal circulation. Presently, unfractionated heparin and LMWH are administered subcutaneously or by intravenous injection. Because of their hydrophilicity, large molecular size, and high-density negative charge, both unfractionated heparin and LMWH exhibit low oral bioavailabilities when administered using conventional oral formulations. In order to evaluate the potential benefits of orally administering unfractionated heparin or LMWH using a formulation of the of the present invention, three different formulations according to the present invention were evaluated using F/L and NF/NL rat models.

In a first study, an in-situ gelling formulation according to the present invention including, by weight percent, 10% unfractionated heparin, 14% sodium caprate, 67.9% CREMOPHOR® EL, and 8.1% propylene glycol laurate was prepared and tested using both an F/L model and a NF/NL model. In both the F/L and NF/NL models, the bioavailablity provided by the in-situ gelling formulation was compared to the bioavailability provided by a saline solution of unfractionated heparin and a i.v. administered dose of unfractionated heparin. In order to assess the bioavailability of unfractionated heparin administered using the in-situ gelling formulation described, the heparin plasma anti-factor Xa activity was measured using ACCUCOLOR (Sigma Diagnostic).

In the F/L model (results shown in FIG. 34), the in-situ gelling formulation provided a Cₘₐₓ (IU/mL) of 10.9, a Tₘₐₓ (h) of 1.3, an AUC (IU*h/mL) of 36.5 and an absolute bioavailability of 61%, while the unfractionated heparin/saline used as the control provided Cₘₐₓ (IU/mL) of 0.6, a Tₘₐₓ (h) of 1.2, an AUC (IU*h/mL) of 0.5 and an absolute bioavailability of 0.8%. The i.v. administered unfractionated heparin provided a Cₘₐₓ (IU/mL) of 7.1, a Tₘₐₓ (h) of 0.1, an AUC (IU*h/mL) of 2.4 and an absolute bioavailability of 100%.

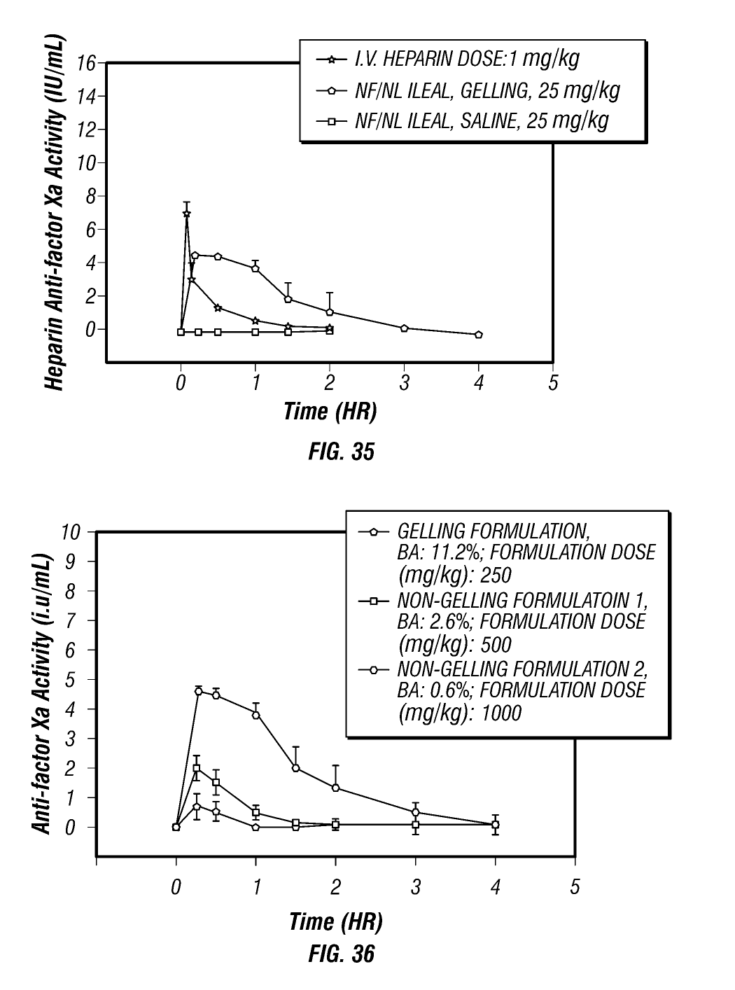

When tested using the NF/NL model (results shown in FIG. 35), the in-situ gelling formulation provided a Cₘₐₓ (IU/mL) of 4.5, a Tₘₐₓ (h) of 0.3, an AUC (IU*h/mL) of 6.7 and an absolute bioavailability of 11%, while the unfractionated heparin/saline used as the control provided Cₘₐₓ (IU/mL) of 0.1, a Tₘₐₓ (h) of 0.7, an AUC (IU*h/mL) of 0.2 and an absolute bioavailability of 0.3%. The i.v. administered unfractionated heparin provided a Cₘₐₓ (IU/mL) of 7.1, a Tₘₐₓ (h) of 0.1, an AUC (IU*h/mL) of 2.4 and an absolute bioavailability of 100%. The reduced bioavailability provided by the in-situ gelling formulation may be attributed in this case to dilution effect in the open compartment model. However, the result is still very encouraging compared to 0.3% of the bioavailability for the control.

In a second study, a second in-situ gelling composition comprising, in weight percent, 10% unfractionated heparin, 14% sodium caprate, and 76% CREMOPHOR® EL was prepared and tested using a NF/NL model. The formulation was mixed homogeneously using either a homogenizer or a mechanical agitator. The doses of the formulation, the unfractionated heparin, and the sodium caprate were, respectively, 250 mg/kg, 25 mg/kg, and 35 mg/kg. The heparin plasma anti-factor Xa activity was again measured using ACCUCOLOR (Sigma Diagnostic), and the bioavailability of unfractionated heparin achieved using this second in-situ gelling formulation was calculated to be 11.2% compared to intravenous injection (shown in FIG. 36). Two non-gelling formulations were prepared and evaluated using the NF/NL model. One comprised, in weight percent, 5.0% unfractionated heparin, 7.0% sodium caprate, 38.0% CREMOPHOR® EL, and 50% water. The other comprised, in weight percent, 2.5% unfractionated heparin, 3.5% sodium caprate, 19.0% CREMOPHOR® EL, and 75% water. To keep the unfractionated heparin dose and sodium caprate dose for the non-gelling formulations the same as that delivered by the second in-situ gelling formulation, the formulation dose of the non-gelling formulations was increased to 500 mg/kg and 1000 mg/kg, correspondingly. As shown in FIG. 36, the bioavailability of unfractionated heparin provided by the two non-gelling formulations was much lower than that achieved using the second in-situ gelling composition.

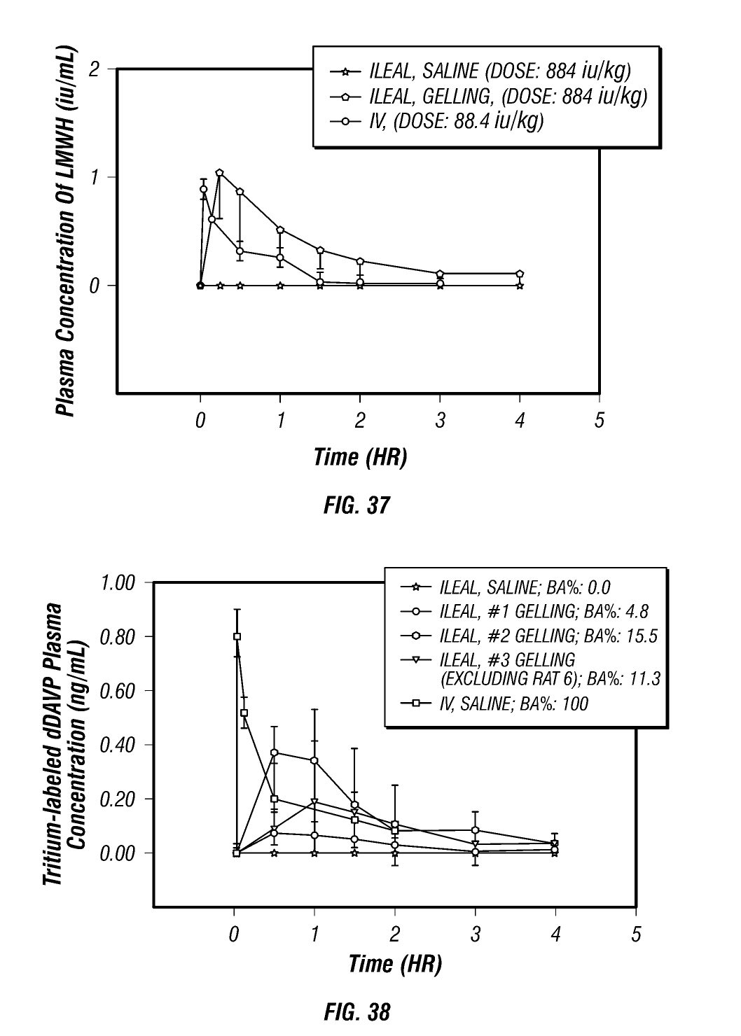

A third study was conducted, wherein an in-situ gelling formulation including, in weight percent, 9.6% LMWH, 28% sodium caprate, and 64.4% CREMOPHOR® EL was prepared and tested using a NF/NL model. A LMWH saline solution was also evaluated as a negative control, and an intravenous injection of LMWH was evaluated as a positive control. LMWH bioavailability was evaluated again by measuring heparin activity using ACCUCOLOR (Sigma Diagnostic). As can be seen by reference to FIG. 37, the i.v. injection provided a Cₘₐₓ (IU/mL) of 0.8, a Tₘₐₓ (h) of 0.03, an AUC (IU*h/mL) of 0.64 and an absolute bioavailability of 100%, the in-situ gelling LMWH formulation provided a Cₘₐₓ (IU/mL) of 1.0, a Tₘₐₓ (h) of 0.25, an AUC (IU*h/mL) of 1.58 and an absolute bioavailability of 24.8%, and the LMWH saline solution provided a Cₘₐₓ (IU/mL) of 0.0, a Tₘₐₓ (h) of N/A, an AUC (IU*h/mL) of 0.00 and an absolute bioavailability of 0% (representing no detectable anti-factor Xa activity).

### EXAMPLE 4

The bioavailability of Desmopressin (dDAVP) administered using formulations according to the present invention was evaluated. dDAVP is a peptide drug used for the treatment of diabetes insipidus, primary nocturnal enuresis, hemophilia, and Type I Von Willebrand's disease. A commercial product providing dDAVP in an oral dosage form is currently indicated for treatment of nocturnal enuresis. However, due to its hydrophilicity and susceptibility to chemical and enzymatic degradation, dDAVP has an extremely low oral bioavailability (about 0.15%). In order to evaluate the potential benefits of orally administering dDAVP using a formulation of the present invention, three different formulations according to the present invention were evaluated using the NF/NL rat model.

FIG. 38 presents the results of a dDAVP bioavailability study conducted using five different formulations, four of which were administered using the NF/NL model. Three of the formulations evaluated were in-situ gelling formulations according to the present invention. The fourth and fifth formulations were provided as a positive and negative control, respectively. The positive control was provided by the intravenous delivery of a dDAVP/saline solution with a dDAVP dose of 2.4 μg/kg (0.4 hot and 2.0 cold). The negative control was administered using the NF/NL model. Each of the formulations was dosed at a formulation dose of 250 mg/kg, and each of the four formulations administered in the NF/NL model provided an ileal dose of 98.3 μg/kg (3.5 μg/kg hot and 94.8 μg/kg cold).

The negative control dDAVP/saline solution included, in weight percent, 0.04% dDAVP and 99.96% saline. As can be seen in FIG. 38, the dDAVP plasma concentration achieved using the negative control was below the detection limit. Therefore, its bioavailability was calculated to be 0.0% compared to intravenous injection (FIG. 38, ileal saline).

The first in-situ gelling formulation included, in weight percent, 0.0394% dDAVP, 71.91% CREMOPHOR® EL, 11.71% lauric acid, 3.01% propylene glycol, 0.02% butylated hydroxytoluene, and 13.31% water. The formulation was mixed homogeneously using either a homogenizer or a mechanical agitator. The dDAVP plasma concentration provided by the first in-situ gelling formulation was measured as a function of time using HPLC with a scintillation counter, and the bioavailability of dDAVP provided by the first in-situ gelling formulation was calculated to be 4.8% compared to intravenous injection (FIG. 38, ileal #1 gelling).

The second in-situ gelling formulation included, in weight percent, 00.0394% dDAVP, 71.91% Tween 80, 11.71% lauric acid, 3.01% propylene glycol, 0.02% butylated hydroxytoluene, and 13.31% water. The formulation was mixed homogeneously using either a homogenizer or a mechanical agitator. The dDAVP plasma concentration provided by the second in-situ gelling formulation was measured as a function of time using HPLC with a scintillation counter, and the bioavailability of dDAVP provided by the second in-situ gelling formulation was calculated to be 15.5% compared to intravenous injection (FIG. 38, ileal #2 gelling).

The third in-situ gelling formulation included, in weight percent, 0.0394% dDAVP, 71.91% Volpos 5, 11.71% lauric acid, 3.01% propylene glycol, 0.02% butylated hydroxytoluene, and 13.31% water. The formulation was mixed homogeneously using either a homogenizer or a mechanical agitator. The dDAVP plasma concentration provided by the third in-situ gelling formulation was measured as a function of time using HPLC with a scintillation counter, and the bioavailability of dDAVP provided by the third in-situ gelling formulation was calculated to be 11.3% compared to intravenous injection (FIG. 38, ileal #3 gelling).

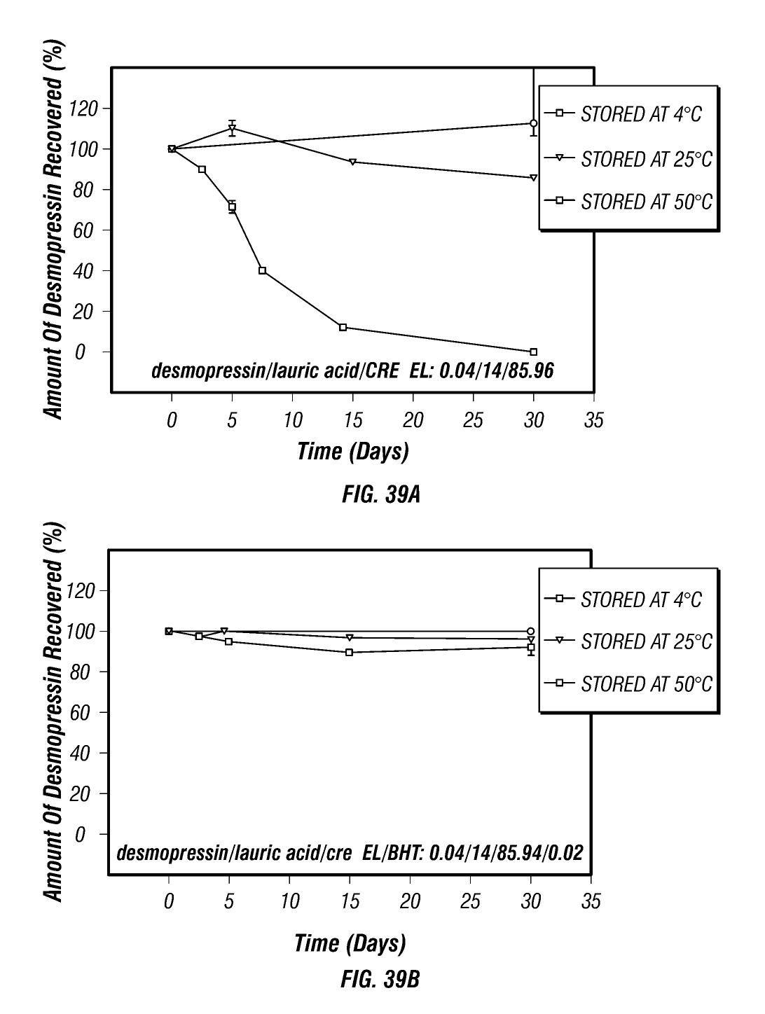

A second study was conducted to evaluate the usefulness of including an antioxidant in a dDAVP formulation of the present invention. For this study two in-situ gelling dDAVP formulations were prepared. The first was prepared without an antioxidant, and the second was prepared with an antioxidant (butylated hydroxytoluene (BHT)). The amounts of each constituent included in both formulations are indicated in FIG. 39. The stability of both formulations was evaluated over the course of 30 days, with samples of each formulation being stored at 4° C., 25° C., and 50° C. during the test period. To assess the stability of the dDAVP over the course of the test, dDAVP periodically recovered from each sample and measured using HPLC. As shown in FIG. 39, the dDAVP included in the formulation including BHT remained stable over the course of the 30 day study, while the dDAVP included in the formulation without BHT shown significant destabilization when stored at 25° C. and 50° C.

Three different dosage forms including an in-situ gelling dDAVP formulation according to the present invention were prepared for a dog oral dosing study. The three different dosage forms included an enteric-coated hard gelatin capsule providing a bolus release of the formulation, and enteric-coated hard gelatin capsule designed to release the in-situ gelling dDAVP formulation at a controlled rate over a 2 hour period, and an enteric-coated hard gelatin capsule designed to release the in-situ gelling dDAVP formulation at a controlled rate over a 4 hour period. Each of the three different dosage forms were loaded with 0.55 g of the in-situ gelling dDAVP formulation, which included, by weight percent, 0.036% desmopressin acetate, 83.372% Tween 80, 13.572% lauric acid, 3.0% propylene glycol, and 0.02% BHT. The dosage forms compared in the study were orally administered to beagles that were food fasted overnight.

The in-situ gelling dDAVP formulation was prepared by heating the Tween 80 to 50° C. and dissolving the lauric acid in the Tween 80. The BHT was then dissolved in the Tween 80/lauric acid solution at room temperature. A separate solution was prepared by dissolving desmopressin acetate into the propylene glycol. Appropriate amounts of both solutions were then weighed and combined to form the in-situ gelling dDAVP formulation.

The enteric-coated, hard gelatin capsule providing a bolus release of formulation was prepared by first providing a clear, elongated “0” hydroxypropylmethylcellulose (HPMC) capsule was provided. The capsule was separated into a body and a cap, and the body was filled with 0.55 g of the in-situ gelling dDAVP formulation. After filling, the body was capped and sealed with an etOH solution consisting of 7% solid pvp K29-32/klucel: 70/30. A banding machine was used in the sealing process. A 12″ Hi coater was used to coat the filled and sealed capsule with and enteric membrane (eudragit L100-55/TEC:70/30) of about 150 mg.

To prepare the enteric-coated, controlled release capsules, clear, elongated “0” HPMC capsules were provided and separated into bodies and caps. The bodies of the capsules were filled with 0.55 g of the in-situ gelling dDAVP formulation and an osmotic engine tablet composed of an Na CMC push and micro fine wax barrier was positioned on top of the in-situ gelling formulation within the bodies, with the micro fine wax barrier of the osmotic engine tablets in contact with the in-situ gelling dDAVP formulation. Caps were then positioned on the filled bodies and the seams of the filled capsules were sealed with a banding machine. The sealing solution included 7% solid pvp k29-32/klucel:70/30 in EtOH. Capsules providing 2 hour controlled release of the formulation were produced by coating filled and sealed capsules with a CA 398-10/pluronic F68:70//30 membrane having a membrane weight of about 50 mg, while capsules providing 4 hour controlled release of the formulation were produced by coating filled and sealed capsules with about a CA 398-10/pluronic F68:70//30 membrane having a membrane weight of about 100 mg. Both the 2-hour controlled release capsules and the 4-hour controlled release capsules were coated with enteric membranes having membrane weights of about 110 mg and comprising eudragit L100-55/TEC:70/30. Drilling an exit orifice in each capsule using a mechanical drill completed the controlled release dosage forms. The diameter of the exit orifice provided in each capsule was about 8-9 mil.

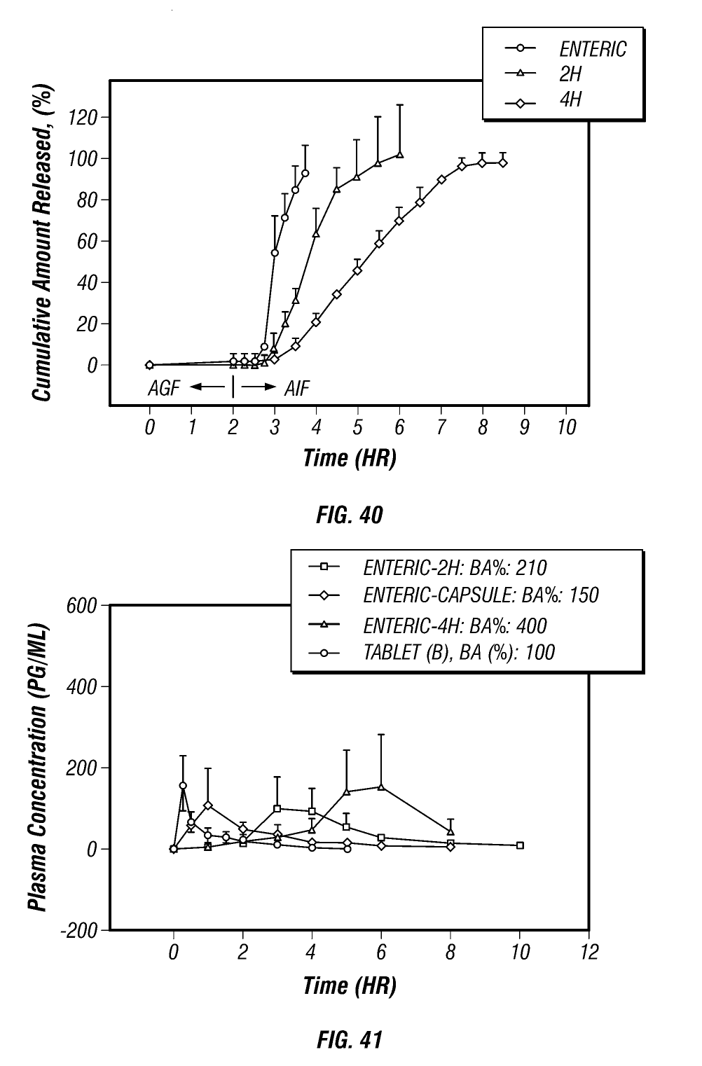

FIG. 40 provides a graph illustrating the in-vitro release profiles provided by each of the dosage forms produced. Each of the dosage forms was placed in artificial gastric fluid for 2 hours and then transferred to artificial intestinal fluid for the duration of the test. The release profile achieved by the enteric coated dosage form providing a bolus dose of the in-situ gelling dDAVP formulation is labeled “enteric” in FIG. 40, while the release profiles achieved by the enteric coated dosage forms designed for 2 hour and 4 hour controlled release of the in-situ gelling dDAVP formulation are labeled “2h” and “4h”, respectively.

The plasma levels (measured using IRA with a lower detection limit of 4.0 pg/ml) and oral bioavailability of dDAVP achieved in the fasted dogs using the prepared dosage forms are described in the graph provided in FIG. 41. As can be appreciated by reference to FIG. 41, the plasma levels and oral bioavailabilities achieved by each of the three dosage forms delivering the in-situ gelling dDAVP formulation were compared to the oral bioavailability achieved by a commercial dDAVP tablet (“Tablet (B)”). The dDAVP plasma concentration and bioavailability achieved by the enteric coated dosage form providing a bolus dose of the in-situ gelling formulation is labeled as “Enteric-Capsule”, while the dDAVP plasma concentration and bioavailability achieved by the enteric coated dosage forms providing controlled release of the in-situ gelling dDAVP formulation over 2 hours and 4 hours, are labeled as “Enteric-2h” and “Enteric-4-h”, respectively. Each of the three dosage forms delivering the in-situ gelling dDAVP formulation achieved bioavailabilities which were greater than the commercial dDAVP tablet, with the dosage form providing controlled release of the in-situ gelling dDAVP formulation over 4 hours resulting in a bioavailability four times greater than the commercial dDAVP tablet.

What is claimed is:

1. A formulation for increasing the bioavailability of an orally administered hydrophilic macromolecule, the formulation comprising a hydrophilic macromolecule, a permeation enhancer, and a carrier capable of forming a bioadhesive gel, the formulation being formulated such that the formulation is released within the gastrointestinal tract as a liquid and forms a bioadhesive gel in-situ after the formulation has had some opportunity to spread across the surface of the gastrointestinal mucosal membrane.

2. The formulation of claim 1, wherein the hydrophilic macromolecule comprises a polypeptide.

3. The formulation of claim 2, wherein the polypeptide is selected from a group consisting of insulin, human growth hormone, IFN-α, samon calcitonin, erythropoietin (EPO), TPA (Activase), G-CSF (Neupogen), Factor VIII (Kogenate), growth hormone-releasing peptide, β-casomorphine, renin inhibitor, tetragastrin, pepstatinylglycine, leuprolide, empedopeptin, β-lactoglobulin, TRH analogues, ACE inhibitors, and cyclosporine.

4. The formulation of claim 1, wherein the hydrophilic macromolecule comprises a polysaccharide.

5. The formulation of claim 4, wherein the polysaccharide is selected from a group consisting of pentosan polysulfate sodium (PPS), unfractionated heparin, and low molecular weight heparin (LMWH).

6. The formulation of claim 1, wherein the permeation enhancer comprises a fatty acid permeation enhancer.

7. The formulation of claim 1, wherein the permeation enhancer is selected from a group consisting of ethylene-diamine tetra-acetic acid (EDTA), bile salt permeation enhancers, fatty acid permeation enhancers, acyl carnitines, and salicylates.

8. The formulation of claim 1, wherein the carrier comprises a nonionic surfactant.

9. The formulation of claim 8, wherein the nonionic surfactant is selected from a group consisting of CREMOPHOR® EL, CREMOPHOR® RH, Incordas 30, polyoxyethylene castor oil, polyethylene 9 castor oil, polyethylene 15 castor oil, d-α-tocopheryl polyethylene glycol succinate (TPGS), myverol, oleth-3, oleth-5, polyoxyl 10 oleyl ether, oleth-20, steareth-2, stearteth-10, steareth-20, ceteareth-20, polyoxyl 20, cetostearyl ether, PPG-5 ceteth-20, PEG-6 capryl/capric triglyceride, PLURONIC® L10, L31, L35, L42, L43, L44, L62, L61, L63, L72, L81, L101, L121, and L122, TWEEN 20, TWEEN 40, TWEEN 60, TWEEN 65, TWEEN 80, TWEEN 81, TWEEN 85, PEG 20 almond glycerides, PEG-60 almond glycerides, PEG-20 corn glycerides, and PEG-60 corn glycerides.

10. The formulation of claim 1, wherein the formulation further comprises a viscosity reducing agent.

11. The formulation of claim 10, wherein the viscosity reducing agent is selected from group consisting of polyoxyethylene 5 castor oil, polyoxyethylene 9 castor oil, labratil, labrasol, capmul GMO (glyceryl mono oleate), capmul MCM (medium chain mono- and diglyceride), capmul MCM C8 (glyceryl mono caprylate), capmul MCM C10 (glyceryl mono caprate), capmul GMS-50 (glyceryl mono stearate), caplex 100 (propylene glycol didecanoate), caplex 200 (propylene glycol dicaprylate/dicaprate), caplex 800 (propylene glycol di 2-ethyl hexanoate), captex 300 (glyceryl tricapryl/caprate), captex 1000 (glyceryl tricaprate), captex 822 (glyceryl triandecanoate), captex 350 (glyceryl tricaprylate/caprate/laurate), caplex 810 (glyceryl tricaprylate/caprate/linoleate), capmul PG8 (propylene mono caprylate), propylene glycol, and propylene glycol laurate (PGL).

12. The formulation of claim 1, wherein the formulation further comprises an antioxidant.

13. The formulation of claim 12, wherein the antioxidant is selected from a group consisting of butylated hydroxytoluene, ascorbic acid, fumaric acid, malic acid, ∝-tocopherol, ascorbic acid palmitate, butylated hydroxyanisole, propyl gallate, sodium ascorbate, and sodium metabisulfate.

14. A formulation for enhancing the bioavailability of an orally administered hydrophilic macromolecule, the formulation comprising a hydrophilic macromolecule, a permeation enhancer, and a carrier capable of forming a bioadhesive gel, wherein the hydrophilic macromolecule comprises between about 0.01 wt % and about 50 wt % of the formulation, the permeation enhancer comprises between about 11% and about 30% of the formulation, and the carrier comprising between about 35% and 88% of the formulation.

15. The formulation of claim 14, wherein the hydrophilic macromolecule, the permeation enhancer, and carrier are included in amounts that allow the formulation to be released within the gastrointestinal tract as a liquid before forming a bioadhesive gel in-situ after the formulation has had some opportunity to spread across a surface of a gastrointestinal mucosal membrane.

16. A dosage form comprising:
a formulation comprising a hydrophilic macromolecule, a permeation enhancer, and a carrier capable of forming a bioadhesive gel, the formulation being formulated such that the formulation is released within the gastrointestinal tract as a liquid and forms a bioadhesive gel in-situ after the formulation has had some opportunity to spread across a surface of a gastrointestinal mucosal membrane; and
a delivery device configured to release the formulation within the gastrointestinal tract of a subject at a controlled rate over a period of time.

17. The dosage form of claim 16, wherein the delivery device is provided with an enteric coating.

18. The dosage form of claim 16, wherein the delivery device comprises:
a capsule;
a deformable barrier layer formed on the gelatin capsule;
an osmotic layer formed on the barrier layer; and
a semipermeable membrane formed over the semipermeable membrane.

19. The dosage form of claim 16, wherein the delivery device comprises:
a capsule having an interior compartment, the interior compartment containing the formulation, an osmotic engine, and a barrier layer positioned between the formulation and the osmotic engine; and
a semipermeable membrane.

20. A controlled release dosage form comprising:
a liquid formulation comprising a hydrophilic macromolecule, the formulation being capable of enhancing the oral bioavailability of the hydrophilic macromolecule; and
a delivery device configured to deliver the formulation over a desired period of time.
